SEQUENCE LISTING (169 sequences)

SEQ ID NO: 1 - protein, 429 aa, synthetic construct
MGPFCLGCSHRKCYSPIRNLISQETFKFHFKNLGYAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQNLCRLVQEGAQVAAMDLYEFKKCWKKFVDNGGRRFRPWKRLLTNFRYQDSKLQEILRPCYISVPSSSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 2 - protein, 107 aa, synthetic construct
YAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQ

SEQ ID NO: 3 - protein, 107 aa, synthetic construct
DAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQ

SEQ ID NO: 4 - protein, 107 aa, synthetic construct
EAKGRKDTFLCYEVTRKDCDSPVSLHHGVFKNKDNIHAEICFLYWFHDKVLKVLSPREEFKITWYMSWSPCFECAEQIVRFLATHHNLSLDIFSSRLYNVQDPETQQ

SEQ ID NO: 5 - protein, 107 aa, synthetic construct
YAKGRHDTFLCYEVTRKDCDSPVSLHHGVFKNKDNIHAEICFLYWFHDKVLKVLSPREEFKITWYMSYSPCFECAEQIVRFLATHHNLSLDIFSSRLYNVQDPETQQ

SEQ ID NO: 6 - protein, 222 aa, synthetic construct
SSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 7 - protein, 223 aa, synthetic construct
MSSSTLSNICLTKGLPETRFWVEGRRMDPLSEEEFYSQFYNQRVKHLCYYHRMKPYLCYQLEQFNGQAPLKGCLLSEKGKQHAEILFLDKIRSMELSQVTITCYLTWSPCPNCAWQLAAFKRDRPDLILHIYTSRLYFHWKRPFQKGLCSLWQSGILVDVMDLPQFTDCWTNFVNPKRPFWPWKGLEIISRRTQRRLRRIKESWGLQDLVNDFGNLQLGPPMS

SEQ ID NO: 8 - protein, 164 aa, synthetic construct
MKPHFRNTVERMYRDTFSYNFYNRPILSRRNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRLSQAGARVKIMDDEEFAYCWENFVYSEG

SEQ ID NO: 9 - protein, 166 aa, synthetic construct
MNPQIRNPMERMYRDTFYDNFENEPILYGRSYTWLCYEVKIKRGRSNLLWDTGVFRGQVYFKPQYHAEMCFLSWFCGNQLPAYKCFQITWFVSWTPCPDCVAKLAEFLSEHPNVTLTISAARLYYYWERDYRRALCRLSQAGARVKIMDYEEFAYCWENFVYNEGQ

SEQ ID NO: 10 - protein, 119 aa, synthetic construct
MGESLFKGPRDYNPISSTICHLTNESDGHTTSLYGIGFGPFIITNKHLFRRNNGTLLVQSLHGVFKVKNTTTLQQHLIDGRDMIIIRMPKDFPPFPQKLKFREPQREERICLVTTNFQT

SEQ ID NO: 11 - protein, 116 aa, synthetic construct
MKSMSSMVSDTSCTFPSSDGIFWKHWIQTKDGQCGSPLVSTRDGFIVGIHSASNFTNTNNYFTSVPKNFMELLTNQEAQQWVSGWRLNADSVLWGGHKVFMVKPEEPFQPVKEATQ

SEQ ID NO: 12 - protein, 7 aa, synthetic construct
ENLYFQS

SEQ ID NO: 13 - protein, 246 aa, synthetic construct
MASSNSMFRGLRDYNPISNNICHLTNVSDGASNSLYGVGFGPLILTNRHLFERNNGELVIKSRHGEFVIKNTTQLHLLPIPDRDLLLIRLPKDVPPFPQKLGFRQPEKGERICMVGSNFQTKSITSIVSETSTIMPVENSQFWKHWISTKDGQCGSPMVSTKDGKILGLHSLANFQNSINYFAAFPDDFAEKYLHTIEAHEWVKHWKYNTSAISWGSLNIQASQPSGLFKVSKLISDLDSTAVYAQ

SEQ ID NO: 14 - protein, 7 aa, synthetic construct
GGCSHQS

SEQ ID NO: 15 - protein, 246 aa, synthetic construct
MASSKSLFRGLRDYNPIASSICQLNNSSGARQSEMFGLGFGGLIVTNQHLFKRNDGELTIRSHHGEFVVKDTKTLKLLPCKGRDIVIIRLPKDFPPFPRRLQFRTPTTEDRVCLIGSNFQTKSISSTMSETSATYPVDNSHFWKHWISTKDGHCGLPIVSTRDGSILGLHSLANSTNTQNFYAAFPDNFETTYLSNQDNDNWIKQWRYNPDEVCWGSLQLKRDIPQSPFTICKLLTDLDGEFVYTQ

SEQ ID NO: 16 - protein, 8 aa, synthetic construct
QVVVHQSK

SEQ ID NO: 17 - protein, 247 aa, synthetic construct
MASAKSLMRGLRDFNPIAQTVCRLKVSVEYGASEMYGFGFGAYIVANHHLFRSYNGSMEVQSMHGTFRVKNLHSLSVLPIKGRDIILIKMPKDFPVFPQKLHFRAPTQNERICLVGTNFQEKYASSIITETSTTYNIPGSTFWKHWIETDNGHCGLPVVSTADGCIVGIHSLANNAHTTNYYSAFDEDFESKYLRTNEHNEWVKSWVYNPDTVLWGPLKLKDSTPKGLFKTTKLVQDLIDHDVVVEQ

SEQ ID NO: 18 - protein, 8 aa, synthetic construct
YDVRHQSR

SEQ ID NO: 19 - protein, 239 aa, synthetic construct
MASDMYIERAGDITWEKDAEVTGNSPRLDVALDESGDFSLVEEDGPPMREGGGGSGGGGSGALWDVPAPKEVKKGETTDGVYRVMTRRLLGSTQVGVGVMQEGVFHTMWHVTKGAALRSGEGRLDPYWGDVKQDLVSYCGPWKLDAAWDGLSEVQLLAVPPGERARNIQTLPGIFKTKDGDIGAVALDYPAGTSGSPILDKCGRVIGLYGNGVVIKNGSYVSAITQGKREEETPVECFE

SEQ ID NO: 20 - protein, 8 aa, synthetic construct
KERKRRGA

SEQ ID NO: 21 - protein, 243 aa, synthetic construct
MASSTDMWIERTADISWESDAEITGSSERVDVRLDDDGNFQLMNDPGAPWKGGGGSGGGGGVLWDTPSPKEYKKGDTTTGVYRIMTRGLLGSYQAGAGVMVEGVFHTLWHTTKGAALMSGEGRLDPYWGSVKEDRLCYGGPWKLQHKWNGQDEVQMIVVEPGKNVKNVQTKPGVFKTPEGEIGAVTLDFPTGTSGSPIVDKNGDVIGLYGNGVIMPNGSYISAIVQGERMDEPIPAGFEPEML

SEQ ID NO: 22 - protein, 8 aa, synthetic construct
KQKKRGGK

SEQ ID NO: 23 - RNA, 19 nt, synthetic construct
  source 1..19: mol_type = other RNA
acatgaggatcacccatgt

SEQ ID NO: 24 - RNA, 144 nt, synthetic construct
  source 1..144: mol_type = other RNA
gtttgagagctaggccattacatgaggatcacccatgtttctgcagggcctagcaagttcaaataaggctagtccgttatcaacttgttgccaacatgaggatcacccatgtctgcagggccttaagtggcaccgagtcggtgc

SEQ ID NO: 25 - RNA, 25 nt, synthetic construct
  source 1..25: mol_type = other RNA
ggagcagacgatatggcgtcgctcc

SEQ ID NO: 26 - RNA, 122 nt, synthetic construct
  source 1..122: mol_type = other RNA
gtttgagagctaccggagcagacgatatggcgtcgctccggtagcaagttcaaataaggctagtccgttatcaacttggagcagacgatatggcgtcgctccaagtggcaccgagtcggtgc

SEQ ID NO: 27 - RNA, 15 nt, synthetic construct
  source 1..15: mol_type = other RNA
gccctgaagaagggc

SEQ ID NO: 28 - RNA, 106 nt, synthetic construct
  source 1..106: mol_type = other RNA
gtttgagagctagggccctgaagaagggccctagcaagttcaaataaggctagtccgttatcaacttgggccctgaagaagggcccaagtggcaccgagtcggtgc

SEQ ID NO: 29 - protein, 117 aa, synthetic construct
MASNFTQFVLVDNGGTGDVTVAPSNFANGIAEWISSNSRSQAYKVTCSVRQSSAQNRKYTIKVEVPKGAWRSYLNMELTIPIFATNSDCELIVKAMQGLLKDGNPIPSAIAANSGIY

SEQ ID NO: 30 - protein, 123 aa, synthetic construct
MGSKTIVLSVGEATRTLTEIQSTADRQIFEEKVGPLVGRLRLTASLRQNGAKTAYRVNLKLDQADVVDSGLPKVRYTQVWSHDVTIVANSTEASRKSLYDLTKSLVATSQVEDLVVNLVPLGR

SEQ ID NO: 31 - protein, 23 aa, synthetic construct
MGNARTRRRERRAEKQAQWKAAN

SEQ ID NO: 32 - protein, 83 aa, synthetic construct
TNLSDIIEKETGKQLVIQESILMLPEEVEEVIGNKPESDILVHTAYDESTDENVMLLTSDAPEYKPWALVIQDSNGENKIKML

SEQ ID NO: 33 - protein, 22 aa, synthetic construct
GSGATNFSLLKQAGDVEENPGP

SEQ ID NO: 34 - protein, 21 aa, synthetic construct
GSGEGRGSLLTCGDVEENPGP

SEQ ID NO: 35 - protein, 23 aa, synthetic construct
GSGQCTNYALLKLAGDVESNPGP

SEQ ID NO: 36 - DNA, 587 bp, synthetic construct
  source 1..587: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtacacatgctttacatgtgtttagtcgaggttaaaaaaacgtctaggccccccgaaccacggggacgtggttttcctttgaaaaacacgatgataatatggccacaacc

SEQ ID NO: 37 - protein, 74 aa, synthetic construct
SEKGKQHAEILFLDKIRSMELSQVTITCYLTWSPCPNCAWQLAAFKRDRPDLILHIYTSRLYFHWKRPFQKGLC

SEQ ID NO: 38 - protein, 74 aa, synthetic construct
SEKGKQHAEILFLDKIRSMELSQVTITCYLTWSPCPNCAWQLAAFKRDRPDLIPHIYTSRLYFHWKRPFQKGLC

SEQ ID NO: 39 - protein, 74 aa, synthetic construct
SEKGKQHAEILFLDKIRSMELSQVTITCYLTWSPCPNCAWRLAAFKRDRPDLILHIYTSRLYFHWKRPFQKGLC

SEQ ID NO: 40 - protein, 74 aa, synthetic construct
SEKGKQHAEILFLNKIRSMELSQVTITCYLTWSPCPNCAWQLAAFKKDRPDLILHIYTSRLYFHWKRPFQKGLC

SEQ ID NO: 41 - protein, 74 aa, synthetic construct
SKKGKQHAEILFLDKIRSMELSQVTITCYLTWSPCPNCAWQLAAFKRDHPDLILHIYTSRLYFHWKRPFQKGLC

SEQ ID NO: 42 - protein, 74 aa, synthetic construct
SKKGKQHAEILFLEKIRSMELSQMRITCYLTWSPCPNCAWQLAAFQKDRPDLILHIYTSRLYFHWRRIFQKGLC

SEQ ID NO: 43 - protein, 74 aa, synthetic construct; identical to SEQ ID NO: 42

SEQ ID NO: 44 - protein, 74 aa, synthetic construct
SKKGKQHAEILFLDKIRSMELSQVRITCYLTWSPCPNCAWQLETFKKDRPDLILHIYTSRLYFHWKRAFQEGLC

SEQ ID NO: 45 - protein, 75 aa, synthetic construct
SKKGKPHAEILFLDKMWSMEELSQVRITCYLTWSPCPNCARQLAAFKKDHPGLILRIYTSRLYFYWRRKFQKGLC

SEQ ID NO: 46 - protein, 73 aa, synthetic construct
KKGEQHVEILFLEKMRSMELSQVRITCYLTWSPCPNCARQLAAFKKDHPDLILRIYTSRLYFYWRKKFQKGLC

SEQ ID NO: 47 - protein, 74 aa, synthetic construct
SKKGRQHAEILFLEKVRSMQLSQVRITCYLTWSPCPNCAWQLAAFKMDHPDLILRIYASRLYFHWRRAFQKGLC

SEQ ID NO: 48 - protein, 73 aa, synthetic construct
NKKGKHAEILFIDEMRSLELGQVQITCYLTWSPCPNCAQELAAFKSDHPDLVLRIYTSRLYFHWRRKYQEGLC

SEQ ID NO: 49 - protein, 73 aa, synthetic construct
NKKGKHAEILFIDEMRSLELGQARITCYLTWSPCPNCAQKLAAFKKDHPDLVLRVYTSRLYFHWRRKYQEGLC

SEQ ID NO: 50 - protein, 73 aa, synthetic construct
NKKDKHAEILFIDKMRSLELCQVRITCYLTWSPCPNCAQELAAFKKDHPDLVLRIYTSRLYFHWRRKYQEGLC

SEQ ID NO: 51 - protein, 73 aa, synthetic construct
NKKGKHAEILFIDEMRSLKLSQERITCYLTWSPCPNCAQELAAFKRDHPGLVLRIYASRLYFHWRRKYQEGLC

SEQ ID NO: 52 - protein, 72 aa, synthetic construct
NKRAKHAEILLIDMMRSMELGQVQITCYITWSPCPTCAQELAAFKQDHPDLVLRIYASRLYFHWKRKFQKGL

SEQ ID NO: 53 - protein, 73 aa, synthetic construct
NKKGRHAEICLIDEMRSLGLGKAQITCYLTWSPCRKCAQELATFKKDHPDLVLRVYASRLYFHWSRKYQQGLC

SEQ ID NO: 54 - protein, 75 aa, synthetic construct
NKKGHHAEIRFIERIRSMGLDPSQDYQITCYLTWSPCLDCAFKLAKLKKDFPRLTLRIFTSRLYFHWIRKFQKGL

SEQ ID NO: 55 - protein, 72 aa, synthetic construct
NKKGKHAEARFVDKMRSMQLDHALITCYLTWSPCLDCSQKLAALKRDHPGLTLRIFTSRLYFHWVKKFQEGL

SEQ ID NO: 56 - protein, 76 aa, synthetic construct
SPQKGHHAESRFIKRISSMDLDRSRSYQITCFLTWSPCPSCAQELASFKRAHPHLRFQIFVSRLYFHWKRSYQAGL

SEQ ID NO: 57 - protein, 74 aa, synthetic construct
KKGYHAESRFIKRICSMDLGQDQSYQVTCFLTWSPCPHCAQELVSFKRAHPHLRLQIFTARLFFHWKRSYQEGL

SEQ ID NO: 58 - protein, 74 aa, synthetic construct
KKGQHAEIRFIERIHSMALDQARSYQITCFLTWSPCPFCAQELASFKSTHPRVHLQIFVSRLYFHWKRSYQEGL

SEQ ID NO: 59 - protein, 75 aa, synthetic construct
NKKGHHAEIRFIKKIRSLDLDQSQNYEVTCYLTWSPCPDCAQELVALTRSHPHVRLRLFTSRLYFHWFWSFQEGL

SEQ ID NO: 60 - protein, 72 aa, synthetic construct
NRHAEICFIDEIESMGLDKTQCYEVTCYLTWSPCPSCAQKLAAFTKAQVHLNLRIFASRLYYHWRSSYQKGL

SEQ ID NO: 61 - protein, 72 aa, synthetic construct
NRHAEICFIDEIESMGLDKTQCYEVTCYLTWSPCPSCAQKLVAFAKAQDHLNLRIFASRLYYHWRRRYKEGL

SEQ ID NO: 62 - protein, 70 aa, synthetic construct
HVEICFIDKIASMELDKTQCYDVTCYLTWSPCPSCAQKLAAFAKAQDHLNLRIFASRLYYHWRRSYQKGL

SEQ ID NO: 63 - protein, 75 aa, synthetic construct
NKKKCHAEICFINEIKSMGLDETQCYQVTCYLTWSPCSSCAWELVDFIKAHDHLNLGIFASRLYYHWCKPQQKGL

SEQ ID NO: 64 - protein, 76 aa, synthetic construct
ENKKKCHAEICFINEIKSMGLDETQCYQVTCYLTWSPCSSCAWELVDFIKAHDHLNLGIFASRLYYHWCKPQQKGL

SEQ ID NO: 65 - protein, 75 aa, synthetic construct
NKKKCHAEICFINEIKSMGLDETQCYQVTCYLTWSPCSSCAWKLVDFIQAHDHLNLRIFASRLYYHWCKPQQEGL

SEQ ID NO: 66 - protein, 75 aa, synthetic construct
NKKKRHAEIRFINKIKSMGLDETQCYQVTCYLTWSPCPSCAWELVDFIKAHDHLNLGIFASRLYYHWCRHQQEGL

SEQ ID NO: 67 - protein, 75 aa, synthetic construct
NKKKDHAEIRFINKIKSMGLDETQCYQVTCYLTWSPCPSCAGELVDFIKAHRHLNLRIFASRLYYHWRPNYQEGL

SEQ ID NO: 68 - protein, 75 aa, synthetic construct
NKKKEHAEIRFINKIKSMGLDETQCYQVTCYLTWSPCPSCAGKLVDFIKAHHHLNLRIFASRLYYHWRPNYQEGL

SEQ ID NO: 69 - protein, 75 aa, synthetic construct
NKKKHHAEIHFINKIKSMGLDETQCYQVTCYLTWSPCPSCARELVDFIKAHRHLNLRIFASRLYYHWRPHYQEGL

SEQ ID NO: 70 - protein, 75 aa, synthetic construct
NKKQRHAEIRFIDKINSLDLNPSQSYKIICYITWSPCPNCANELVNFITRNNHLKLEIFASRLYFHWIKPFKMGL

SEQ ID NO: 71 - protein, 75 aa, synthetic construct
NKKQRHAEIRFIDKINSLDLNPSQSYKIICYITWSPCPNCASELVDFITRNDHLDLQIFASRLYFHWIKPFKRGL

SEQ ID NO: 72 - protein, 75 aa, synthetic construct
NKKQRHAEIRFIDKINSLNLDRRQSYKIICYITWSPCPRCASELVDFITGNDHLNLQIFASRLYFHWKKPFQRGL

SEQ ID NO: 73 - protein, 75 aa, synthetic construct
NKKKRHAEIRFIDKINSLNLDQNQCYRIICYVTWSPCHNCAKELVDFISNRHHLSLQLFASRLYFHWVRCYQRGL

SEQ ID NO: 74 - protein, 75 aa, synthetic construct
NKKKRHAEIRFIDKIKSLGLDRVQSYEITCYITWSPCPTCALELVAFTRDYPRLSLQIFASRLYFHWRRRSIQGL

SEQ ID NO: 75 - protein, 75 aa, synthetic construct
NKKKRHAEIRFIDKINSLGLDQDQSYEITCYVTWSPCATCACKLIKFTRKFPNLSLRIFVSRLYYHWFRQNQQGL

SEQ ID NO: 76 - protein, 74 aa, synthetic construct
KKKRHAEIRFIDSIRALQLDQSQRFEITCYLTWSPCPTCAKELAMFVQDHPHISLRLFASRLYFHWRWKYQEGL

SEQ ID NO: 77 - protein, 74 aa, synthetic construct
KKKRHAEIRFIDNIKALRLDTSQRFEITCYVTWSPCPTCAKELVAFVRDHRHISLRLFASRLYFHWLRENKKGL

SEQ ID NO: 78 - protein, 75 aa, synthetic construct
NKKKRHAEIRFIDKIRSLQRDSSQTFEITCYVTWSPCFTCAEELVAFVRDHPHVRLRLFASRLYFHWLRKYQEGL

SEQ ID NO: 79 - protein, 75 aa, synthetic construct
NKKKRHAEICFIDKIKSLTRDTSQRFEIICYITWSPCPFCAEELVAFVKDNPHLSLRIFASRLYVHWRWKYQQGL

SEQ ID NO: 80 - protein, 75 aa, synthetic construct; identical to SEQ ID NO: 79

SEQ ID NO: 81 - protein, 75 aa, synthetic construct
NKKHRHAEVRFIAKIRSMSLDLDQKHQLTCYLTWSPCPSCAQELVTFMAESRHLNLQVFVSRLYFHWQRDFQQGL

SEQ ID NO: 82 - protein, 108 aa, synthetic construct
RRNTVWLCYEVKTKGPSRPRLDTKIFRGQVYFEPQYHAEMCFLSWFCGNQLPAYKCFQITWFVSWTPCPDCVAKLAEFLAEHPNVTLTISAARLYYYWERDYRRALCR

SEQ ID NO: 83 - protein, 108 aa, synthetic construct
RRNTVWLCYEVKTKGPSRPRLDTKIFRGQVYFQFENHAEMCFLSWFCGNQLPAYKCFQITWFVSWTPCPDCVAKLAEFLAEHPNVTLTISAARLYYYWERDYRRALCR

SEQ ID NO: 84 - protein, 108 aa, synthetic construct
RRNTVWLCYEVKTRGPSMPTWGAKIFRGQVYFEPQYHAEMCFLSWFCGNQLPAYKCFQITWFVSWTPCPDCVGKVAEFLAEHPNVTLTISAARLYYYWETDYRRALCR

SEQ ID NO: 85 - protein, 108 aa, synthetic construct
RRNTVWLCYEVKTRGPSMPTWDTKIFRGQVYSKPEHHAEMCFLSRFCGNQLPAYKRFQITWFVSWTPCPDCVAKVAEFLAEHPNVTLTISAARLYYYWETDYRRALCR

SEQ ID NO: 86 - protein, 108 aa, synthetic construct
RRNTVWLCYEVKTRGPSVPTWGTKIFRGQVYSKPEHHAEMCFLSWFCGNQLPTYKRFQITWFVSWTPCPDCVAKVAEFLAEHPNVTLTISAARLYYYWETDYRRALCR

SEQ ID NO: 87 - protein, 108 aa, synthetic construct
RRNTVWLCYEVKTRGPSMPTWGAKIFRGQVYFEPQYHAEMCFLSWFCGNQLPAYKRFQITWFVSWTPCPDCVAKVAEFLAEHPNVTLTISAARLYYYWETDYRRALCR

SEQ ID NO: 88 - protein, 108 aa, synthetic construct; identical to SEQ ID NO: 87

SEQ ID NO: 89 - protein, 108 aa, synthetic construct; identical to SEQ ID NO: 87

SEQ ID NO: 90 - protein, 108 aa, synthetic construct
RRNTVWLCYEVKTRGPSMPTWDTKIFRGQVYSKPEHHAEMCFLSRFCGNQLPAYKRFQITWFVSWTPCTDCVAKVAEFLAEHPNVTLTISAARLYYYWETDYRRALCR

SEQ ID NO: 91 - protein, 108 aa, synthetic construct
RRNTVWLCYEVKTRGPSMPTWGAKIFRGQVYFEPQYHAEMCFLSWFCGNQLPAYKRFRITWFVSWTPCPDCVAKVAEFLAEHPNVTLTISAARLYYYWETDYRRALCR

SEQ ID NO: 92 - protein, 99 aa, synthetic construct
RRNTVWLCYEVKTKGPSRPPLDAKIFRGQVYFEPQYHAEMCFLSWFCGNQLPAYKCFQITWFVSWTPCPDCVAKLAEFLAEHPNVTLTISAARLYYYWE

SEQ ID NO: 93 - protein, 108 aa, synthetic construct
RRNTVWLCYEVKTRGPSMPTWDAKIFRGQVYFQPQYHAEMCFLSRFCGNQLPAYKRFQITWFVSWTPCPDCVVKVTEFLAEHPNVTLTISAARLYYYWETDYRRALCR

SEQ ID NO: 94 - protein, 108 aa, synthetic construct
RRNTVWLCYKVKTKGPSRPPLNAKIFRGQVYFEPQYHAEMCFLSWFCGNQLSAYERFQITWFVSWTPCPDCVAMLAEFLAEHPNVTLTVSAARLYYYWERDYRGALRR

SEQ ID NO: 95 - protein, 108 aa, synthetic construct
RRNTVWLCYEVKTRGPSMPTWGTKIFRGQVCFEPQYHAEMCFLSRFCGNQLPAYKRFQITWFVSWTPCPDCVAKVAEFLAEHPNVTLTISAARLYYYWETDYRRALCR

SEQ ID NO: 96 - protein, 108 aa, synthetic construct; identical to SEQ ID NO: 95

SEQ ID NO: 97 - protein, 108 aa, synthetic construct
RSYTWLCYEVKIKRGRSNLLWDTGVFRGQVYFEPQYHAEMCFLSWFCGNQLPAYKCFQITWFVSWTPCPDCVAKLAEFLSEHPNVTLTISAARLYYYWERDYRRALCR

SEQ ID NO: 98 - protein, 108 aa, synthetic construct
RSYNWLCYEVKIKRGRSNLLWNTGVFRGQMYSQPEHHAEMCFLSWFCGNQLPAYKCFQITWFVSWTPCPDCVAKLAEFLAEYPNVTLTISAARLYYYWERDYRRALCR

SEQ ID NO: 99 - protein, 108 aa, synthetic construct
RSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCR

SEQ ID NO: 100 - protein, 108 aa, synthetic construct
RRNTVWLCYEVKTRGPSMPTWGTKIFRGQVYFQPQYHAEMCFLSRFCGNQLPAYKRFQITWFVSWNPCPDCVAKVIEFLAEHPNVTLTISAARLYYYWGRDWRRALRR

SEQ ID NO: 101 - protein, 102 aa, synthetic construct
RRNTVWLCYKVKTRGPSMPTWGTKIFRGQVYFQPQYHAEMCFLSWFCGNQLPAYKRFQITWFVSWTPCPDCVVKVAEFLAEHPNVTLTISAARLYYYWETDY

SEQ ID NO: 102 - protein, 108 aa, synthetic construct
RSYTWLCYEVKIKRGRSNLLWDTGVFRGQMYSQPEHHAEMCFLSWFCGNQLPAYKCFQITWFVSWTPCLDCVAKLAEFLAEYPNVTLTISTARLYYYWERDYRRALCR

SEQ ID NO: 103 - protein, 108 aa, synthetic construct
RSYTWLCYEVKIRRGHSNLLWDTGVFRGQMYSQPEHHAEMYFLSWFCGNQLSAYKCFQITWFVSWTPCPDCVAKLAEFLAEHPNVTLTISAARLYYYWERDYRRALCR

SEQ ID NO: 104 - protein, 108 aa, synthetic construct
RSYTWLCYEVKIRKDPSKLPWDTGVFRGQMYFQPEYHAEMCFLSWFCGNQLPAYKRFQITWFVSWTPCPDCVAKVAEFLAEHPNVTLTISAARLYYYWEKDWQRALCR

SEQ ID NO: 105 - protein, 108 aa, synthetic construct
RRNTVWLCYEVKTKDPSRPRLDTKIFRGKVYFQLENHAEMCFLSWFCGNQLPANRCFQITWFVSWNPCLPCVAKVTKFLAEHPNVTLTISAARLYYYRARDWRRALRR

SEQ ID NO: 106 - protein, 108 aa, synthetic construct
RSYTWLCYEVKIRKDPSKLPWDTGVFRGQMYSKPEHHAEMCFLSWFCGNQLPAHKRFQITWFVSWTPCPDCVAKVAEFLAEYPNVTLTISAARLYYYWETDYRRALCR

SEQ ID NO: 107 - protein, 108 aa, synthetic construct; identical to SEQ ID NO: 106

SEQ ID NO: 108 - protein, 109 aa, synthetic construct
RRSYTWLCYEVKIRKDPSKLPWDTGVFRGQMYFQPEYHAEMCFLSWFCGNQLPAYKRFQITWFVSWTPCPDCVAKVAVFLAEHPNVTLTISAARLYYYWEKDWQRALCR

SEQ ID NO: 109 - protein, 108 aa, synthetic construct
RSYTWLCYEVKIRKDPSKLPWDTGVFRGQVYSEPEHHAEMYFLSWFCGNQLPAYKRFWITWFVSWTPCPDCVAKLAEFLTEHPNVTLTISAARLYYYRGRDWRRALCR

SEQ ID NO: 110 - protein, 108 aa, synthetic construct
RSYTWLCYEVKKRKDPSKLPWDTGVFRGQVYSEPEHHAEMYFLSWFCGNQLPAYKRFWITWFVSWTPCPDCVAKVAEFLAEHPKVTLTISAARLYYYWDRDWRRALCR

SEQ ID NO: 111 - protein, 108 aa, synthetic construct
RSYTWLCYEVKIRKDPSKLPWDTGVFRGQVYSEPEHHAEMYFLSWFCGNQLPAYKRFQITWFVSWTPCPDCVAKVAEFLTEHPNVTLTISAARLYYYRGRDWRRALCR

SEQ ID NO: 112 - protein, 108 aa, synthetic construct; identical to SEQ ID NO: 111

SEQ ID NO: 113 - protein, 100 aa, synthetic construct
RRNYTWLCYEVKIRKDPSKLAWDTGVFRGQVYSQPEHHAEMCFLSWFCGNQLSAYERFQITWFVSWTPCPDCVAKLAEFLAEHPNVTLTVSAARLYYYWE

SEQ ID NO: 114 - protein, 108 aa, synthetic construct
RSYTWLCYEVKIRKDPSKLPWDTGVFRGQVYSKPEHHAEMCFLSRFCGNQLPAYKRFQITWFVSWNPCPDCVAKVIEFLAEHPNVTLTISTARLYYYWGRDWQRALCR

SEQ ID NO: 115 - protein, 108 aa, synthetic construct
RSYTWLCYEVKIRKDPSKLPWDTGVFRGQVYSKPEHHAEMCFLSRFCGNQLPAYKRFQITWFVSWNPCPDCVVKVIEFLAEHPNVTLTISTARLYYYWGRDWQRALCR

SEQ ID NO: 116 - protein, 108 aa, synthetic construct
RSYTWLCYEVKIRKDPSKLPWDTGVFRGQVYSKPEHHAEMCFLSRFCGNQLPAYKRFQITWFVSWNPCPDCVAKVTEFLAEHPNVTLTISTARLYYYWGRDWQRALCR

SEQ ID NO: 117 - protein, 108 aa, synthetic construct
RSYTWLCYEVKIRKDPSKLPWDTGVFRGQVYFQPQYHAEMCFLSWFCGNQLPAYKRFQITWFVSWNPCPDCVAKVTEFLAEHPNVTLTISVARLYYYRGKDWRRALCR

SEQ ID NO: 118 - protein, 121 aa, synthetic construct
RRNYTWLCYEVKIRKDPSKLAWDTGVFRGQVLPKLQSNHRREVYFEPQYHAEMCFLSWFCGNQLSAYERFQITWFVSWTPCPDCVAMLAEFLAEHPNVTLTVSAARLYYYWERDYRGALRR

SEQ ID NO: 119 - protein, 120 aa, synthetic construct
RRYTWLCYEVKIKKDPSKLPWDTGVFQGQVRPKFQSNRRYEVYFQPQYHAEMCFLSWFCGNQLPAYKHFQITWFVSWNPCPDCVAKVTEFLAEHPNVTLTISAARLYYYWGKDWRRALCR

SEQ ID NO: 120 - protein, 79 aa, synthetic construct
MYSQPEHHAEMCFLSWFCGNQLSAYKCFQITWFVSWTPCPDCVAKLAKFLAEHPNVTLTISAARLYYYWERDYRRALCR

SEQ ID NO: 121 - protein, 120 aa, synthetic construct
RSYTWLCYEVKIRKDPSKLPWDTGVFRGQVRPKLQSNRRYEVYFQPQYHAEMCFLSWFCGNQLPAYKRFQITWFVSWNPCPDCVAKVTEFLAEHPNVTLTISAARLYYYWGKDWRRALRR

SEQ ID NO: 122 - protein, 108 aa, synthetic construct
RRYTWLCYEVKIMKDHSKLPWYTGVFRGQVYFEPQNHAEMCFLSWFCGNQLPAYECCQITWFVSWTPCPDCVAKVTEFLAEHPNVTLTISAARLYYYRGRDWRRALRR

SEQ ID NO: 123 - protein, 108 aa, synthetic construct
RSYTWLCYEVKIRKDPSKLPWYTGVFRGQVYFQPQYHAEMCFLSWFCGNQLPANKRFQITWFVSWNPCPDCVAKVTEFLAEHPNVTLTISVARLYYYRGKDWRRALRR

SEQ ID NO: 124 - protein, 108 aa, synthetic construct
RSYTWLCYEVKIRKDPSKLPWDTGVFRDQVYFQPQYHAEMCFLSWFCGNQLPANKRFQITWFVSWNPCPDCVTKVTEFLAEHPNVTLTISVARLYYYRGKDWRRALRR

SEQ ID NO: 125 - protein, 120 aa, synthetic construct
RRYTWLCYEVKIKKDPSKLPWDTGVFPGQVRPKFQSNRRYEVYFQPQYHAEMYFLSWFCGNQLPAYKHFQITWFVSWNPCPDCVAKVTEFLAEHRNVTLTISAARLYYYWGKDWRRALCR

SEQ ID NO: 126 - protein, 129 aa, synthetic construct
RSYTWLCYEVKIRKDPSKLPWDTGVFRGQVRPKLQSNRRYELSNWECRKHVYFQPQYHAEMCFLSWFCGNQLPANKRFQITWFVSWNPCPDCVAKVTEFLAEHPNVTLTISAARLYYYWGKDWRRALRR

SEQ ID NO: 127 - protein, 110 aa, synthetic construct
GRSYNWLCYEVKIKRGRSNLLWNTGVFRGQMYSQPEHHAEMCFLSWFCGNQLPAYKCFQITWFVSWTPCPDCVAKLAEFLAEYPNVTLTISTARLYYYWERDYRRALCRL

SEQ ID NO: 128 - protein, 110 aa, synthetic construct
GRSYTWLCYEVKIRRGHSNLLWDTGVFRGQMYSQPEHHAEMYFLSWFCGNQLPAYKCFQITWFVSWTPCPDCVAKLAEFLAEHPNVTLTISAARLYYYWERDYRRALCRL

SEQ ID NO: 129 - protein, 110 aa, synthetic construct
GRSYTWLCYEVKIRRGHSNLLWDTGVFRGQMYSQPEHHAEMCFLSWFCGNQLSAYKCFQITWFVSWTPCPDCVAKLAKFLAEHPNVTLTISAARLYYYWERDYRRALCRL

SEQ ID NO: 130 - protein, 98 aa, synthetic construct
RNTVWLCYEVKTKGPSRPPLDAKIFRGQVYFEPQYHAEMCFLSWFCGNQLPAYKCFQITWFVSWTPCPDCVAKLAEFLAEHPNVTLTISAARLYYYWE

SEQ ID NO: 131 - protein, 108 aa, synthetic construct
RNTVWLCYEVKTKGPSRPRLDTKIFRGQVYFEPQYHAEMCFLSWFCGNQLPAYKCFQITWFVSWTPCPDCVAKLAEFLAEHPNVTLTISAARLYYYWERDYRRALCRL

SEQ ID NO: 132 - protein, 108 aa, synthetic construct
RNTVWLCYEVKTKGPSRPRLDAKIFRGQVYSQPEHHAEMCFLSWFCGNQLPAYKCFQITWFVSWTPCPDCVAKLAEFLAEHPNVTLTISAARLYYYWERDYRRALCRL

SEQ ID NO: 133 - protein, 108 aa, synthetic construct
RNTVWLCYEVKTRGPSMPTWGTKIFRGQVCFEPQYHAEMCFLSRFCGNQLPAYKRFQITWFVSWTPCPDCVAKVAEFLAEHPNVTLTISAARLYYYWETDYRRALCRL

SEQ ID NO: 134 - protein, 108 aa, synthetic construct; identical to SEQ ID NO: 133

SEQ ID NO: 135 - protein, 108 aa, synthetic construct
RNTVWLCYEVKTRGPSMPTWGAKIFRGQVYFEPQYHAEMCFLSWFCGNQLPAYKRFQITWFVSWTPCPDCVAKVAEFLAEHPNVTLTISAARLYYYWETDYRRALCRL

SEQ ID NO: 136 - protein, 101 aa, synthetic construct
RNTVWLCYKVKTRGPSMPTWGTKIFRGQVYFQPQYHAEMCFLSWFCGNQLPAYKRFQITWFVSWTPCPDCVVKVAEFLAEHPNVTLTISAARLYYYWETDY

SEQ ID NO: 137 - protein, 108 aa, synthetic construct
RNTVWLCYEVKTRGPSMPTWDTKIFRGQVYSKPEHHAEMCFLSRFCGNQLPAYKRFQITWFVSWTPCPDCVAKVAEFLAEHPNVTLTISAARLYYYWETDYRRALCRL

SEQ ID NO: 138 - protein, 108 aa, synthetic construct
RNTVWLCYEVKTRGPSMPTWGTKIFRGQVYFQPQYHAEMCFLSRFCGNQLPAYKRFQITWFVSWNPCPDCVAKVIEFLAEHPNVTLTISAARLYYYWGRDWRRALRRL

SEQ ID NO: 139 - protein, 110 aa, synthetic construct
GRSYTWLCYEVKIRKDPSKLPWYTGVFRGQVYSKPEHHAEMCFLSRFCGNQLPAYKRFQITWFVSWNPCPDCVAKVIEFLAEHPNVTLTISAARLYYYWSRDWQRALCRL

SEQ ID NO: 140 - protein, 110 aa, synthetic construct
GRSYTWLCYEVKIRKDPSKLPWDTGVFRGQVYSKPEHHAEMCFLSRFCGNQLPAYKRFQITWFVSWNPCPDCVAKVIEFLAEHPNVTLTISTARLYYYWGRDWQRALCRL

SEQ ID NO: 141 - protein, 110 aa, synthetic construct; identical to SEQ ID NO: 140

SEQ ID NO: 142 - protein, 110 aa, synthetic construct
GRSYTWLCYEVKIRKDPSKLPWDTGVFRGQVYSKPEHHAEMCFLSRFCGNQLPAYKRFQITWFVSWNPCPDCVVKVIEFLAEHPNVTLTISTARLYYYWGRDWQRALCRL

SEQ ID NO: 143 - protein, 110 aa, synthetic construct
GRSYTWLCYEVKIRKDPSKLPWYTGVFRGQVYSKPEHHAEMCFLSRFCGNQLPAYKRFQITWFVSWNPCPDCVAKVIEFLAEHPNVTLTIFTARLYYYWGRDWQRALCRL

SEQ ID NO: 144 - protein, 110 aa, synthetic construct
GRSYTWLCYEVKIRKDPSKLPWDTGVFRGQVYSKPEHHAEMCFLSRFCGNQLPAYKRFQITWFVSWNPCPDCVAKVTEFLAEHPNVTLTISTARLYYYWGRDWQRALCRL

SEQ ID NO: 145 - protein, 110 aa, synthetic construct
GRSYTWLCYEVKIRKDPSKLPWDTGVFRGQVYSEPEHHAEMYFLSWFCGNQLPAYKRFQITWFVSWTPCPDCVAKVAEFLTEHPNVTLTISAARLYYYRGRDWRRALCRL

SEQ ID NO: 146 - protein, 110 aa, synthetic construct; identical to SEQ ID NO: 145

SEQ ID NO: 147 - protein, 110 aa, synthetic construct
GRSYTWLCYEVKIRKDPSKLPWDTGVFRGQMYSKPEHHAEMCFLSWFCGNQLPAHKRFQITWFVSWTPCPDCVAKVAEFLAEYPNVTLTISAARLYYYWETDYRRALCRL

SEQ ID NO: 148 - protein, 109 aa, synthetic construct
RSYTWLCYEVKIRKDPSKLPWDTGVFRGQMYFQPEYHAEMCFLSWFCGNQLPAYKRFQITWFVSWTPCPDCVAKVAVFLAEHPNVTLTISAARLYYYWEKDWQRALCRL

SEQ ID NO: 149 - protein, 110 aa, synthetic construct
GRSYTWLCYEVKIKKYPSKLLWDTGVFQGQVYFQPQYHAEMCFLSRFCGNQLPAYKRFQITWFVSWNPCPDCVAKVTEFLAEHPNVTLTISAARLYYYWEKDXRRALRRL

SEQ ID NO: 150 - protein, 110 aa, synthetic construct
GRSYTWLCYEVKIKEDPSKLLWDTGVFQGQVYFQPQYHAEMCFLSRFCGNQLPAYKRFQITWFVSWNPCPDCVAKVTEFLAEHPNVTLTISAARLYYYWGRDWRRALRRL

SEQ ID NO: 151 - protein, 122 aa, synthetic construct
GRRYTWLCYEVKIKKDPSKLPWDTGVFPGQVRPKFQSNRRYEVYFQPQYHAEMYFLSWFCGNQLPAYKHFQITWFVSWNPCPDCVAKVTEFLAEHRNVTLTISAARLYYYWGKDWRRALCRL

SEQ ID NO: 152 - protein, 106 aa, synthetic construct
GRRYTWLCYEVKIKKDPSKLPWDTGVFPGQPQYHAEMYFLSWFCGNQLPAYKHFQITWFVSWNPCPDCVAKVTEFLAEHRNVTLTISAARLYYYWGKDWRRALCRL

SEQ ID NO: 153 - protein, 122 aa, synthetic construct
GRRYTWLCYEVKIKKDPSKLPWDTGVFPGQVRPKFQSNRRQKVYFQPQYHAEMYFLSWFCGNQLPAYKHFQITWFVSWNPCPDCVAKVTEFLAEHRNVTLTISAARLYYYWGKDWRRALCRL

SEQ ID NO: 154 - protein, 122 aa, synthetic construct
GRRYTWLCYEVKIKKDPSKLPWDTGVFQGQVRPKFQSNRRYEVYFQPQYHAEMCFLSWFCGNQLPAYKHFQITWFVSWNPCPDCVAKVTEFLAEHPNVTLTISAARLYYYWGKDWRRALCRL

SEQ ID NO: 155 - protein, 131 aa, synthetic construct
GRSYTWLCYEVKIRKDPSKLPWDTGVFRGQVRPKLQSNRRYELSNWECRKRVYFQPQYHAEMYFLSWFCGNQLPANKRFQITWFASWNPCPDCVAKVTEFLAEHPNVTLTISVARLYYYRGKDWRRALRRL

SEQ ID NO: 156 - protein, 110 aa, synthetic construct
GRSYTWLCYEVKIRKDPSKLPWDTGVFRGQVYFQPQYHAEMYFLSWFCGNQLPANKRFQITWFASWNPCPDCVAKVTEFLAEHPNVTLTISVARLYYYRGKDWRRALRRL

SEQ ID NO: 157 - protein, 110 aa, synthetic construct
GRSYTWLCYEVKIRKDPSKLPWDTGVFRDQVYFQPQYHAEMCFLSWFCGNQLPANKRFQITWFVSWNPCPDCVTKVTEFLAEHPNVTLTISVARLYYYRGKDWRRALRRL

SEQ ID NO: 158 - protein, 110 aa, synthetic construct
GRSYTWLCYEVKIRKDPSKLPWYTGVFRGQVYFQPQYHAEMCFLSWFCGNQLPANKRFQITWFVSWNPCPDCVAKVTEFLAEHPNVTLTISVARLYYYRGKDWRRALRRL

SEQ ID NO: 159 - protein, 110 aa, synthetic construct
GRSYTWLCYEVKIRKDPSKLPWDTGVFRGQVYFQPQYHAEMCFLSWFCGNQLPAYKRFQITWFVSWNPCPDCVAKVTEFLAEHPNVTLTISVARLYYYRGKDWRRALCRL

SEQ ID NO: 160 - protein, 122 aa, synthetic construct
GRSYTWLCYEVKIRRGSSNLLWNTGVFRGPVPPKLQSNHRQEVYFQFENHAEMCFLSWFCGNRLPANRRFQITWFVSWNPCLPCVVKVTKFLAEHPNVTLTISAARLYYYRDREWRRVLRRL

SEQ ID NO: 161 - protein, 122 aa, synthetic construct
GRSYTWLCYEVKIKRGCSNLIWDTGVFRGPVLPKLQSNHRQEVYFQFENHAEMCFFSWFCGNRLPANRRFQITWFVSWNPCLPCVVKVTKFLAEHPNVTLTISAARLYYYQDREWRRVLRRL

SEQ ID NO: 162 - protein, 122 aa, synthetic construct; identical to SEQ ID NO: 161

SEQ ID NO: 163 - protein, 122 aa, synthetic construct
GRSYTWLCYEVKIKRGRSNLLWDTGVFRGPVLPKRQSNHRQEVYFRFENHAEMCFLSWFCGNRLPANRRFQITWFVSWNPCLPCVVKVTKFLAEHPNVTLTISAARLYYYRDRDWRWVLLRL

SEQ ID NO: 164 - protein, 122 aa, synthetic construct
GRSYTWLCYEVKIRKDPSKLPWDKGVFRGQVLPKFQSNHRQEVYFQLENHAEMCFLSWFCGNQLPANRRFQITWFVSWNPCLPCVAKVTEFLAEHPNVTLTISAARLYYYRGRDWRRALRRL

SEQ ID NO: 165 - protein, 110 aa, synthetic construct
GKKYTWLCYEVKIKKDTSKLPWNTGVFRGQVNFNPEHHAEMYFLSWFRGKLLPACKRSQITWFVSWNPCLYCVAKVAEFLAEHPNVTLTVSTARLYCYWKKDWRRALRKL

SEQ ID NO: 166 - protein, 110 aa, synthetic construct; identical to SEQ ID NO: 165

SEQ ID NO: 167 - protein, 110 aa, synthetic construct
GRRYTWLCYEVKIMKDHSKLPWYTGVFRGQVYFEPQNHAEMCFLSWFCGNQLPAYECCQITWFVSWTPCPDCVAKVTEFLAEHPNVTLTISAARLYYYRGRDWRRALRRL

SEQ ID NO: 168 - protein, 110 aa, synthetic construct
GRRYTWLCYEVKISKDPSKLPWDTGIFRGQVYFEPQYHAEMCFLSWYCGNQLPAYKCFQITWFVSWTPCPDCVGKVAEFLAEHPNVTLTISAARLYYYWETDYRRALCRL

SEQ ID NO: 169 - protein, 121 aa, synthetic construct
RNYTWLCYEVKIRKDPSKLAWDTGVFRGQVLPKLQSNHRREVYFEPQYHAEMCFLSWFCGNQLSAYERFQITWFVSWTPCPDCVAMLAEFLAEHPNVTLTVSAARLYYYWERDYRGALRRL

(19) United States
(12) Patent Application Publication
(10) Pub. No.: US 2025/0154490 A1
(43) Pub. Date: May 15, 2025

(54) MUTANT CYTIDINE DEAMINASES WITH IMPROVED EDITING PRECISION

(71) Applicant: CORRECTSEQUENCE THERAPEUTICS, Shanghai (CN)

(72) Inventors: Lijie WANG, Shanghai (CN); Tao WEI, Shanghai (CN); Yichuan WANG, Shanghai (CN); Xiaodun MOU, Shanghai (CN)

(21) Appl. No.: 18/837,561

(22) PCT Filed: Feb. 17, 2023
(86) PCT No.: PCT/CN2023/076923

§ 371 Date: Aug. 9, 2024

(30) Foreign Application Priority Data
Feb. 17, 2022 (WO) PCT/CN2022/076699

(51) Int. Cl. C12N 9/78; C12N 9/22; C12N 15/11

(52) U.S. Cl. CPC C12N 9/78; C12N 9/22; C12N 15/111; C07K 2319/50; C12N 2310/20; C12Y 305/04

(57) ABSTRACT

Provided are mutant cytidine deaminases and related molecules useful for conducting base editing with reduced or no off-target mutations and with improved editing site precision. The mutant catalytic domain of the mouse APOBEC3 protein includes one or more mutations which helps to narrow the editing window while maintaining high editing efficiency. Example mutations include Y35D and K40H-W102Y. Also provided are improved base editing systems and methods using these mutant cytidine deaminases.

### BACKGROUND

The combination of CRISPR-Cas9 and cytidine deaminases leads to cytosine base editors (CBEs) for programmable cytosine to thymine (C-to-T) substitution, which has been applied to achieve efficient editing in various species successfully and holds great potentials in clinical applications. As the base editing process does not depend on the generation of DNA double strand break (DSB), unwanted nucleotide insertions/deletions (indels) or DNA damage responses (DDRs) can be largely avoided.

The transformer base editor (tBE) system contains a cytidine deaminase inhibitor (dCDI) domain and a split-TEV protease (see, e.g., WO2020156575). Thus, tBE remains inactive at off-target sites with a cleavable fusion of dCDI domain and eliminates unintended off-target mutations. Only when binding at on-target sites, is tBE transformed to cleave off the dCDI domain and catalyzes targeted deamination for precise editing. Specifically, tBE uses a sgRNA (normally 20 nt) to bind at the target genomic site and a helper sgRNA (hsgRNA, normally 10 to 20 nt) to bind at a nearby region (preferably upstream to the target genomic site). The binding of two gRNAs can guide the components of tBE system to correctly assemble at the target genomic site for base editing. tBE can specifically edit cytosine in target regions with no observable off-target mutations.

### SUMMARY

The present disclosure, in some embodiments, provides mutant cytidine deaminases and related molecules useful for conducting base editing with reduced or no off-target mutations and with improved editing site precision. The mutant catalytic domain (mA3CDA1) of the mouse APOBEC3 protein includes one or more mutations which helps to narrow the editing window while maintaining high editing efficiency. Example mutations include Y35D and K40H-W102Y. Also provided are improved prime editing systems and methods using these mutant cytidine deaminases.

According to one embodiment of the present disclosure, provided is a protein, comprising a catalytic domain of a mutant mouse APOBEC3 protein, wherein the catalytic domain has at least 85% sequence identity to amino acid residues 35-141 of SEQ ID NO: 1 and comprises a substitution, relative to SEQ ID NO: 1, at a residue selected from the group consisting of Y35, K37, R39, K40, N66, W102, Y132, and combinations thereof.

In some embodiments, the substitution is selected from the group consisting of:

| Residue | Substitution |
|---|---|
| Y35 | D or E |
| K37 | D or E |
| R39 | A, G, I, L or V |
| K40 | A, G, I, L, V or H |
| N66 | G, A, I, L, V or Q |
| W102 | Y or F, and |
| Y132 | F or W. |

In some embodiments, the catalytic domain retains the amino acids of SEQ ID NO: 1 at residues H71 and E73. In some embodiments, the catalytic domain retains the amino acids of SEQ ID NO: 1 at residues D41, F43, F64, A72, P104, C105 and C108. In some embodiments, the substitution is selected from the group consisting of Y35D, Y35E, K37D, R39A, K40A, K40H, N66A, N66G, N66Q, W102Y, W102F, Y132F, and combinations thereof.

In some embodiments, the substitution is Y35D or Y35E. In some embodiments, the catalytic domain comprises the amino acid sequence of SEQ ID NO: 3. In some embodiments, the substitution is K40H and W102Y. In some embodiments, the catalytic domain comprises the amino acid sequence of SEQ ID NO: 5.

Also provided is a fusion protein comprising a first fragment comprising the protein of the disclosure, and a second fragment comprising a nucleobase deaminase inhibitor. In some embodiments, the fusion protein further comprises a protease cleavage site between the first fragment and the second fragment. In some embodiments, the nucleobase deaminase inhibitor is an inhibitory domain of a nucleobase deaminase.

In some embodiments, the nucleobase deaminase inhibitor comprises the amino acid sequence of SEQ ID NO: 7, 8 or 9, or amino acids residues 128-223 of SEQ ID NO: 7.

Also provided, in some embodiments, is a dual guide RNA system, comprising: a target single guide RNA comprising a first spacer having sequence complementarity to a target nucleic acid sequence proximate to a first PAM site, a helper single guide RNA comprising a second spacer having sequence complementarity to a second nucleic acid sequence proximate to a second PAM site, a clustered regularly interspaced short palindromic repeats (CRISPR)-associated (Cas) protein, and a protein or a fusion protein of the current disclosure.

In some embodiments, the second PAM site is from 34 to 91 bases from the first PAM site.

Yet another embodiment provides a method for introducing a C-to-T substitution at a cytosine in a target nucleic acid, comprising contacting the target nucleic acid with a CRISPR-associated (Cas) protein, a protein or a fusion protein of the instant disclosure, a single-guide RNA (sgRNA), and a helper single-guide RNA (hsgRNA), wherein the sgRNA and the hsgRNA can hybridize to the target nucleic acid.

In some embodiments, the cytosine is between nucleotide positions 4 and 8 3′ to a protospacer adjacent motif (PAM) sequence on the target nucleic acid sequence. In some embodiments, the cytosine is between nucleotide positions 6 and 8 3′ to a protospacer adjacent motif (PAM) sequence on the target nucleic acid sequence.

In another embodiment, provided is a method for introducing a C-to-T substitution at a cytosine in a target nucleic acid, comprising contacting the target nucleic acid with a CRISPR-associated (Cas) protein, a fusion protein of the present disclosure, a single-guide RNA (sgRNA), and a helper single-guide RNA (hsgRNA), wherein the sgRNA and the hsgRNA can hybridize to the target nucleic acid, wherein cytosine is between nucleotide positions 6 and 8 3′ to a protospacer adjacent motif (PAM) sequence on the target nucleic acid sequence, and wherein the catalytic domain comprises the amino acid sequence of SEQ ID NO: 3.

Still further provided is a method for introducing a C-to-T substitution at a cytosine in a target nucleic acid, comprising contacting the target nucleic acid with a CRISPR-associated (Cas) protein, a fusion protein of the instant disclosure, a single-guide RNA (sgRNA), and a helper single-guide RNA (hsgRNA), wherein the sgRNA and the hsgRNA can hybridize to the target nucleic acid, wherein cytosine is between nucleotide positions 4 and 8 3′ to a protospacer adjacent motif (PAM) sequence on the target nucleic acid sequence, and wherein the catalytic domain comprises the amino acid sequence of SEQ ID NO: 5.

### BRIEF DESCRIPTION OF THE DRAWINGS

FIG. 1 demonstrates the editing efficiencies induced by sgRNA-hVEGFA1/hsgRNA-hVEGFA1 and the tBE variants containing single AA changes. (A): Schematic diagram illustrating the co-transfection of sgRNA-hVEGFA1/hsgRNA-hVEGFA1 with tBE or the tBE variants containing indicated single AA changes. (B): Editing efficiency induced by the original tBE and the tBE variants in (A) with sgRNA-hVEGFA1/hsgRNA-hVEGFA1.

FIG. 2 demonstrates the editing efficiencies induced by sgRNA-hVEGFA1/hsgRNA-hVEGFA1 and the tBE variants containing dual AA changes. (A): Schematic diagram illustrating the co-transfection of sgRNA-hVEGFA1/hsgRNA-hVEGFA1 with tBE or the tBE variants containing indicated dual AA changes. (B): Editing efficiency induced by the original tBE and the tBE variants in (A) with sgRNA-hVEGFA1/hsgRNA-hVEGFA1.

FIG. 3 demonstrates the editing efficiencies induced by tBE-Y35D with sgRNA/hsgRNA pairs targeting various genomic sites. (A): Schematic diagram illustrating the co-transfection of sgRNA/hsgRNA pairs with tBE or tBE-Y35D. (B): Editing efficiency induced by tBE and tBE-Y35D with sgRNA/hsgRNA pairs at the indicated target sites.

FIG. 4 demonstrates the editing efficiencies induced by tBE-K40H-W102Y with sgRNA/hsgRNA pairs targeting various genomic sites. (A): Schematic diagram illustrating the co-transfection of sgRNA/hsgRNA pairs with tBE or tBE-K40H-W102Y. (B): Editing efficiency induced by tBE and tBE-K40H-W102Y with sgRNA/hsgRNA pairs at the indicated target sites.

FIG. 5 demonstrates the editing efficiencies induced by tBE-K40H-W102Y with sgRNA/hsgRNA pairs targeting more genomic sites. (A): Schematic diagram illustrating the co-transfection of sgRNA/hsgRNA pairs with tBE or tBE-K40H-W102Y. (B): Editing efficiency induced by tBE and tBE-K40H-W102Y with sgRNA/hsgRNA pairs at the indicated target sites.

FIG. 6 demonstrates the editing windows of tBE-Y35D and tBE-K40H-W102Y. (A): The major editing window of tBE-Y35D spans from position 6 to 8, counting the protospacer adjacent motif (PAM) distal position in target site as 1. (B): The major editing window of tBE-K40H-W102Y spans from position 4 to 8, counting the protospacer adjacent motif (PAM) distal position in target site as 1. The region between two dashed lines is the major editing window of each tBE.

FIG. 7 demonstrates the editing efficiencies induced by tBE-H71E and tBE-E73A with sgRNA/hsgRNA pairs targeting various genomic sites. (A): Schematic diagram illustrating the co-transfection of sgRNA/hsgRNA pairs with tBE, tBE-H71E or tBE-E73A. (B): Editing efficiency induced by tBE, tBE-H71E and tBE-E73A with sgRNA/hsgRNA pairs at the indicated target sites.

FIG. 8 demonstrates the editing efficiencies induced by sgRNA-hFANCF/hsgRNA-hFANCF or sgRNA-hHBG/hsgRNA-hHBG and the tBE with different types of nCas9-UGI proteins. (A): Schematic diagram illustrating the co-transfection of sgRNA-hFANCF/hsgRNA-hFANCF or sgRNA-hHBG/hsgRNA-hHBG with tBE and different types of nCas9-UGI proteins. (B): Editing efficiency induced by different types of nCas9-UGI proteins and the original tBE in (A) with sgRNA-hFANCF/hsgRNA-hFANCF or sgRNA-hHBG/hsgRNA-hHBG.

FIG. 9 demonstrates the editing induced by sgRNA-hBCL11A/hsgRNA-hBCL11A or sgRNA-hVEGFA2-a/hsgRNA-hVEGFA2-a and the tBE with different types of nCas9-UGI proteins. (A): Schematic diagram illustrating the co-transfection of sgRNA-hBCL11A/hsgRNA-hBCL11A or sgRNA-hVEGFA2-a/hsgRNA-hVEGFA2-a with tBE and different types of nCas9-UGI proteins. (B): Editing efficiency induced by different types of nCas9-UGI proteins and the original tBE in (A) with sgRNA-hBCL11A/hsgRNA-hBCL11A or sgRNA-hVEGFA2-a/hsgRNA-hVEGFA2-a.

FIG. 10 demonstrates the editing efficiencies induced by sgRNA-hCD33-AG-15/hsgRNA-hCD33-AG-15 or sgRNA-hCD123-CGA-6/hsgRNA-hCD123-CGA-6 and the tBE with different types of nCas9-UGI proteins. (A): Schematic diagram illustrating the co-transfection of sgRNA-hCD33-AG-15/hsgRNA-hCD33-AG-15 or sgRNA-hCD123-CGA-6/hsgRNA-hCD123-CGA-6 with tBE and different types of nCas9-UGI proteins. (B): Editing efficiency induced by different types of nCas9-UGI proteins and the original tBE in (A) with sgRNA-hCD33-AG-15/hsgRNA-hCD33-AG-15 or sgRNA-hCD123-CGA-6/hsgRNA-hCD123-CGA-6.

FIG. 11 demonstrates the editing efficiencies induced by sgRNA-hPCSK9-TGG-2/hsgRNA-hPCSK9-TGG-2 or sgRNA-hMSSK1-M-b/hsgRNA-hMSSK1-M-b and the tBE with different types of nCas9-UGI proteins. (A): Schematic diagram illustrating the co-transfection of sgRNA-hPCSK9-TGG-2/hsgRNA-hPCSK9-TGG-2 or sgRNA-hMSSK1-M-b/hsgRNA-hMSSK1-M-b with tBE and different types of nCas9-UGI proteins. (B): Editing efficiency induced by different types of nCas9-UGI proteins and the original tBE in (A) with sgRNA-hPCSK9-TGG-2/hsgRNA-hPCSK9-TGG-2 or sgRNA-hMSSK1-M-b/hsgRNA-hMSSK1-M-b.

FIG. 12 demonstrates the editing efficiencies induced by sgRNA-hHAO1-CAG-2/hsgRNA-hHAO1-CAG-2 or sgRNA-hCD45-CAA-1/hsgRNA-hCD45-CAA-1 and the tBE with different types of nCas9-UGI proteins. (A): Schematic diagram illustrating the co-transfection of sgRNA-hHAO1-CAG-2/hsgRNA-hHAO1-CAG-2 or sgRNA-hCD45-CAA-1/hsgRNA-hCD45-CAA-1 with tBE and different types of nCas9-UGI proteins. (B): Editing efficiency induced by different types of nCas9-UGI proteins and the original tBE in (A) with sgRNA-hHAO1-CAG-2/hsgRNA-hHAO1-CAG-2 or sgRNA-hCD45-CAA-1/hsgRNA-hCD45-CAA-1.

FIG. 13 shows results of analysis of editing efficiencies induced by different sgRNA/hsgRNA pairs and the tBE with different types of nCas9-UGI proteins. (A): Schematic diagram illustrating the co-transfection of different sgRNA/hsgRNA pairs with tBE and different types of nCas9-UGI proteins. (B): Editing efficiency induced by different types of nCas9-UGI proteins and the original tBE in (A) with different sgRNA/hsgRNA pairs at the indicated target sites calculated by EditR analysis. (C): Statistical analysis of normalized editing frequencies at all 10 on-target sites shown in B. (D): Statistical analysis of C/G-to-T/A editing fraction at all 10 on-target sites shown in B.

FIG. 14 demonstrates the editing efficiencies induced by tBE, tBE-IRES-TEVC or tBE-IRES-TEVN with nCas9 and 4 different sgRNA/hsgRNA pairs targeting various genomic sites. (A): Schematic diagram illustrating the co-transfection of sgRNA/hsgRNA pairs with tBE, tBE-IRES-TEVC or tBE-IRES-TEVN. (B): Editing efficiency induced by tBE, tBE-IRES-TEVC or tBE-IRES-TEVN in (A) with sgRNA/hsgRNA pairs at the indicated target sites.

FIG. 15 demonstrates the editing efficiencies induced by tBE, tBE-IRES-TEVC or tBE-IRES-TEVN with nCas9 and 4 different sgRNA/hsgRNA pairs targeting various genomic sites. (A): Schematic diagram illustrating the co-transfection of sgRNA/hsgRNA pairs with tBE, tBE-IRES-TEVC or tBE-IRES-TEVN. (B): Editing efficiency induced by tBE, tBE-IRES-TEVC or tBE-IRES-TEVN in (A) with sgRNA/hsgRNA pairs at the indicated target sites.

FIG. 16 demonstrates the editing efficiencies induced by tBE, tBE-IRES-TEVC or tBE-IRES-TEVN with nCas9 and sgRNA-hPCSK9-TGG-11/hsgRNA-hPCSK9-TGG-11. (A): Schematic diagram illustrating the co-transfection of sgRNA/hsgRNA pairs with tBE, tBE-IRES-TEVC or tBE-IRES-TEVN. (B): Editing efficiency induced by tBE, tBE-IRES-TEVC or tBE-IRES-TEVN in (A) with sgRNA-hPCSK9-TGG-11/hsgRNA-hPCSK9-TGG-11. (C): Editing efficiency induced by tBE, tBE-IRES-TEVC or tBE-IRES-TEVN with nCas9 and different sgRNA/hsgRNA pairs at each target sites calculated by EditR analysis.

FIG. 17 demonstrates the editing efficiencies induced by tBE or tBE-IRES-TEVN with nCas9 and different sgRNA/hsgRNA pairs targeting 3 HBV genomic sites in Lenti-HBV HepG2 stable cell line. (A): Schematic diagram illustrating the co-transfection of sgRNA/hsgRNA pairs with tBE or tBE-IRES-TEVN. (B): Editing efficiency induced by tBE or tBE-IRES-TEVN in (A) with sgRNA/hsgRNA paris at the indicated target sites. (C): Editing efficiency induced by tBE or tBE-IRES-TEVN with nCas9 and different sgRNA/hsgRNA pairs at each target sites calculated by EditR analysis.

FIG. 18 demonstrates the editing efficiencies induced by tBE or tBE-IRES-TEVN with nCas9 and different sgRNA/hsgRNA pairs targeting 3 HBV genomic sites in Lenti-HBV 293 FT stable cell line. (A): Schematic diagram illustrating the co-transfection of sgRNA/hsgRNA pairs with tBE or tBE-IRES-TEVN. (B): Editing efficiency induced by tBE or tBE-IRES-TEVN in (A) with sgRNA/hsgRNA paris at the indicated target sites. (C): Editing efficiency induced by tBE or tBE-IRES-TEVN with nCas9 and different sgRNA/hsgRNA pairs at each target sites calculated by EditR analysis.

FIG. 19 demonstrates the editing efficiencies induced by tBE or tBE-IRES-TEVN with nCas9 and targeting 1 PCSK9 genomic sites in Hepa1-6 cell line. (A): Schematic diagram illustrating the co-transfection of sgRNA-mPCSK9-TGG-3/hsgRNA-mPCSK9-TGG-3 pairs with tBE or tBE-IRES-TEVN in wildtype Hepa1-6 by RNA electroporation. (B): Editing efficiency induced by tBE or tBE-IRES-TEVN in (A) with sgRNA/hsgRNA pairs at the indicated target sites. (C): Editing efficiency induced by tBE or tBE-IRES-TEVN with nCas9 and different sgRNA/hsgRNA pairs at each target sites calculated by EditR analysis.

### DETAILED DESCRIPTION

### Definitions

It is to be noted that the term “a” or “an” entity refers to one or more of that entity; for example, “an antibody,” is understood to represent one or more antibodies. As such, the terms “a” (or “an”), “one or more,” and “at least one” can be used interchangeably herein.

As used herein, the term “polypeptide” is intended to encompass a singular “polypeptide” as well as plural “polypeptides,” and refers to a molecule composed of monomers (amino acids) linearly linked by amide bonds (also known as peptide bonds). The term “polypeptide” refers to any chain or chains of two or more amino acids, and does not refer to a specific length of the product. Thus, peptides, dipeptides, tripeptides, oligopeptides, “protein”, “amino acid chain” or any other term used to refer to a chain or chains of two or more amino acids, are included within the definition of “polypeptide,” and the term “polypeptide” may be used instead of, or interchangeably with any of these terms. The term “polypeptide” is also intended to refer to the products of post-expression modifications of the polypeptide, including without limitation glycosylation, acetylation, phosphorylation, amination, derivatization by known protecting/blocking groups, proteolytic cleavage, or modification by non-naturally occurring amino acids. A polypeptide may be derived from a natural biological source or produced by recombinant technology, but is not necessarily translated from a designated nucleic acid sequence. It may be generated in any manner, including by chemical synthesis.

“Homology” or “identity” or “similarity” refers to sequence similarity between two peptides or between two nucleic acid molecules. Homology can be determined by comparing a position in each sequence which may be aligned for purposes of comparison. When a position in the compared sequence is occupied by the same base or amino acid, then the molecules are homologous at that position. A degree of homology between sequences is a function of the number of matching or homologous positions shared by the sequences. An “unrelated” or “non-homologous” sequence shares less than 40% identity, though preferably less than 25% identity, with one of the sequences of the present disclosure.

A polynucleotide or polynucleotide region (or a polypeptide or polypeptide region) has a certain percentage (for example, 60%, 65%, 70%, 75%, 80%, 85%, 90%, 95%, 98% or 99%) of “sequence identity” to another sequence means that, when aligned, that percentage of bases (or amino acids) are the same in comparing the two sequences. This alignment and the percent homology or sequence identity can be determined using software programs known in the art, for example those described in Ausubel et al. eds. (2007) Current Protocols in Molecular Biology. Preferably, default parameters are used for alignment. One alignment program is BLAST, using default parameters.

The term “an equivalent nucleic acid or polynucleotide” refers to a nucleic acid having a nucleotide sequence having a certain degree of homology, or sequence identity, with the nucleotide sequence of the nucleic acid or complement thereof. A homolog of a double stranded nucleic acid is intended to include nucleic acids having a nucleotide sequence which has a certain degree of homology with or with the complement thereof. In one aspect, homologs of nucleic acids are capable of hybridizing to the nucleic acid or complement thereof. Likewise, “an equivalent polypeptide” refers to a polypeptide having a certain degree of homology, or sequence identity, with the amino acid sequence of a reference polypeptide. In some aspects, the sequence identity is at least about 70%, 75%, 80%, 85%, 90%, 95%, 98%, or 99%. In some aspects, the equivalent polypeptide or polynucleotide has one, two, three, four or five addition, deletion, substitution and their combinations thereof as compared to the reference polypeptide or polynucleotide. In some aspects, the equivalent sequence retains the activity (e.g., epitope-binding) or structure (e.g., salt-bridge) of the reference sequence.

The term “encode” as it is applied to polynucleotides refers to a polynucleotide which is said to “encode” a polypeptide if, in its native state or when manipulated by methods well known to those skilled in the art, it can be transcribed and/or translated to produce the mRNA for the polypeptide and/or a fragment thereof. The antisense strand is the complement of such a nucleic acid, and the encoding sequence can be deduced therefrom.

Mutant Cytidine Deaminase with Narrowed Editing Window

Off-target editing by a genome editing system can cause serious side effects in a target organism and thus should be minimized or avoided. The current genome editing tools such as the CRISPR/Cas9 system, base editors and prime editors, however, are associated with frequent off-target editing.

The instant inventors have developed a new base editing system, transformer base editor (tBE), which can specifically edit cytosine in target regions with no observable off-target mutations. The tBE system combines the conventional cytidine deaminase (or a catalytic domain thereof) with a cleavable cytidine deaminase inhibitor (dCDI). tBE remains inactive at off-target sites, and cleavage of the dCDI at the target site activates the catalytic domain, for precise editing.

In addition to off-target editing, bystander mutations by a base editing system are the unwanted base changes in the editing window. Bystander mutation can compromise the editing site precision of base editing system and should be minimized when precise base editing is performed. Thus, new tBE systems with narrowed editing windows, which can reduce bystander mutation, are required.

A commonly used cytidine deaminase is the mouse APOBEC3 (mA3) protein (Access #: NP_001153887.1). It includes a catalytic portion, mA3CDA1, and an inhibitive portion, mA3CDA2. As shown in Table 1, the CDA1 portion includes residues 35 to 141 (underlined; SEQ ID NO: 2), and the CDA2 portion includes residues 208 to 429 (bold; SEQ ID NO: 6) of SEQ ID NO: 1.

TABLE 1
mAPOBEC3 Sequences

| | | SEQ |
|---|---|---|
| | | ID |
| Name | Sequence | NO: |
| mA3 | 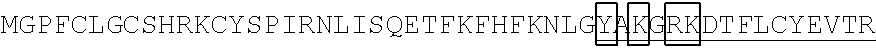 | 1 |
| | 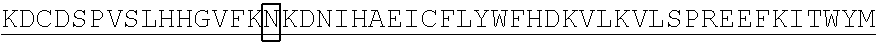 | |
| | 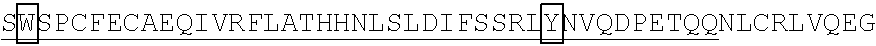 | |
| | AQVAAMDLYEFKKCWKKFVDNGGRRFRPWKRLLTNFRYQDSKLQEILRPC | |
| | YISVPSSSSSTLSNICLTKGLPETRFWVEGRRMDPLSEEEFYSQFYNQRV | |
| | KHLCYYHRMKPYLCYQLEQFNGQAPLKGCLLSEKGKQHAEILFLDKIRSM | |
| | ELSQVTITCYLTWSPCPNCAWQLAAFKRDRPDLILHIYTSRLYFHWKRPF | |
| | QKGLCSLWQSGILVDVMDLPQFTDCWTNFVNPKRPFWPWKGLEIISRRTQ | |
| | RRLRRIKESWGLQDLVNDFGNLQLGPPMS | |
| mA3CDA1 | YAKGRKDTFLCYEVTRKDCDSPVSLHHGVFKNKDNIHAEICFLYWFHDKV | 2 |
| | LKVLSPREEFKITWYMSWSPCFECAEQIVRFLATHHNLSLDIFSSRLYNV | |
| | QDPETQQ | |
| mA3CDA1- | DAKGRKDTFLCYEVTRKDCDSPVSLHHGVFKNKDNIHAEICFLYWFHDKV | 3 |
| Y35D | LKVLSPREEFKITWYMSWSPCFECAEQIVRFLATHHNLSLDIFSSRLYNV | |
| | QDPETQQ | |
| mA3CDA1- | EAKGRKDTFLCYEVTRKDCDSPVSLHHGVFKNKDNIHAEICFLYWFHDKV | 4 |
| Y35E | LKVLSPREEFKITWYMSWSPCFECAEQIVRFLATHHNLSLDIFSSRLYNV | |
| | QDPETQQ | |
| mA3CDA1- | YAKGRHDTFLCYEVTRKDCDSPVSLHHGVFKNKDNIHAEICFLYWFHDKV | 5 |
| K40H- | LKVLSPREEFKITWYMSYSPCFECAEQIVRFLATHHNLSLDIFSSRLYNV | |
| W102Y | QDPETQQ | |
| mA3CDA2 | SSSTLSNICLTKGLPETRFWVEGRRMDPLSEEEFYSQFYNQRVKHLCYYH | 6 |
| | RMKPYLCYQLEQFNGQAPLKGCLLSEKGKQHAEILFLDKIRSMELSQVTI | |
| | TCYLTWSPCPNCAWQLAAFKRDRPDLILHIYTSRLYFHWKRPFQKGLCSL | |
| | WQSGILVDVMDLPQFTDCWTNFVNPKRPFWPWKGLEIISRRTQRRLRRIK | |
| | ESWGLQDLVNDFGNLQLGPPMS | |

Through careful design and testing, the instant inventors discovered that when certain amino acid residues in the mA3CDA1 domain are mutated, the resulting base editors have narrowed editing window while retaining the high editing efficiency. Such amino acid residues include Y35, K37, R39, K40, N66, W102, and Y132. These residues can be individually mutated, or two or more of them can be mutated together. Tested single mutations include Y35D, K37D, R39A, K40A, N66G, W102Y, W102F and Y132F, and tested double mutations include R39A-K40H, R39A-N66A, K40H-W102Y, N66A-W102Y, N66Q-W102Y, K40H-Y132F, N66A-Y132F, N66Q-Y132F, K40A-N66A, K40A-N66Q and K40H-N66G. Additional mutations are also contemplated based on the tested results. For instance, Y35E is contemplated to be similar to Y35D. These mutations (substitutions) are summarized in Table 2 below.

TABLE 2
mAPOBEC3 Substitutions

| Residue | Substitution |
|---|---|
| Y35 | D or E |
| K37 | D or E |
| R39 | A, G, I, L or V |
| K40 | A, G, I, L, V or H |
| N66 | G, A, I, L, V or Q |
| W102 | Y or F |
| Y132 | F or W |

In accordance with one embodiment of the present disclosure, therefore, provided is a mutant mA3CDA1 domain (or a protein that includes the mutant mA3CDA1 domain). In one embodiment, the mutant mA3CDA1 domain is simar to, e.g., having at least 70%, 75%, 80%, 85%, 90%, 95%, 98%, or 99% sequence identity to, the wild-type mA3CDA1 domain. The wild-type mA3CDA1 domain includes amino acid residues 35-141 (SEQ ID NO: 2) of the mouse mA3 protein (SEQ ID NO: 1).

In some embodiments, the mutant mA3CDA1 domain retains the wild-type amino acid residues known to be important to the catalytic activity of the domain. Examples include residues H71 and E73. In some embodiments, the wild-type residues at D41, F43, F64, A72, P104, C105, and C108 are retained.

In some embodiments, the mutant mA3CDA1 domain includes one or more substitutions as shown in Table 2 and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by this particular substitution.

In some embodiments, the mutant mA3CDA1 domain includes substitution Y35D and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by this particular substitution. In some embodiments, this mutant mA3CDA1 domain includes the sequence of SEQ ID NO: 3.

In some embodiments, the mutant mA3CDA1 domain includes substitution Y35E and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by this particular substitution. In some embodiments, this mutant mA3CDA1 domain includes the sequence of SEQ ID NO: 4.

In some embodiments, the mutant mA3CDA1 domain includes substitution K37D (or K37E) and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by this particular substitution.

In some embodiments, the mutant mA3CDA1 domain includes substitution R39A (or R39G, R391, R39L, or R39V) and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by this particular substitution.

In some embodiments, the mutant mA3CDA1 domain includes substitution K40A (or K40G, K40I, K40L, K40V or K40H) and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by this particular substitution.

In some embodiments, the mutant mA3CDA1 domain includes substitution N66G (or N66A, N66I, N66L, N66V or V66Q) and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by this particular substitution.

In some embodiments, the mutant mA3CDA1 domain includes substitution W102Y (or W102F) and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by this particular substitution.

In some embodiments, the mutant mA3CDA1 domain includes substitution Y132F (or Y132W) and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by this particular substitution.

In some embodiments, the mutant mA3CDA1 domain includes substitutions K40H-W102Y and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by these particular substitutions. In some embodiments, this mutant mA3CDA1 domain includes the sequence of SEQ ID NO: 5.

In some embodiments, the mutant mA3CDA1 domain includes substitutions R39A-K40H and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by these particular substitutions.

In some embodiments, the mutant mA3CDA1 domain includes substitutions R39A-N66A and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by these particular substitutions.

In some embodiments, the mutant mA3CDA1 domain includes substitutions N66A-W102Y and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by these particular substitutions.

In some embodiments, the mutant mA3CDA1 domain includes substitutions N66Q-W102Y and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by these particular substitutions.

In some embodiments, the mutant mA3CDA1 domain includes substitutions K40H-Y132F and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by these particular substitutions.

In some embodiments, the mutant mA3CDA1 domain includes substitutions N66A-Y132F and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by these particular substitutions.

In some embodiments, the mutant mA3CDA1 domain includes substitutions N66Q-Y132F and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by these particular substitutions.

In some embodiments, the mutant mA3CDA1 domain includes substitutions K40A-N66A and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by these particular substitutions.

In some embodiments, the mutant mA3CDA1 domain includes substitutions K40A-N66Q and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by these particular substitutions.

In some embodiments, the mutant mA3CDA1 domain includes substitutions K40H-N66G and has at least 70%, 75%, 80%, 85%, 90%, 95%, 98%, or 99% sequence identity to the wild-type mA3CDA1 domain. In some embodiments, this mutant mA3CDA1 domain retains residues H71 and E73 of SEQ ID NO: 1. In some embodiments, this mutant mA3CDA1 domain retains residues D41, F43, F64, A72, P104, C105, and C108 of SEQ ID NO: 1. In some embodiments, this mutant mA3CDA1 domain differs from SEQ ID NO: 2 only by these particular substitutions.

### Fusion Proteins and Base Editors

The mutant mA3CDA1 domains of the instant disclosure can be incorporated into base editors that can be used to achieve precise base editing. In one embodiment, a fusion protein is provided which includes a first fragment that includes a mutant mA3CDA1 domain, and a second fragment that includes a nucleobase deaminase inhibitor. In some embodiments, a protease cleavage site is included in the fusion protein between the first fragment and the second fragment.

A “nucleobase deaminase inhibitor,” accordingly, refers to a protein or a protein domain that inhibits the deaminase activity of a nucleobase deaminase. In some embodiments, the second fragment includes at least an inhibitory core of the inhibitory protein/domain.

Non-limiting example nucleobase deaminase inhibitors include mA3-CDA2, hA3F-CDA1 and hA3B-CDA1 (sequences provided in Table 3), which are the inhibitory domains of the corresponding nucleobase deaminases. Additional nucleobase deaminase inhibitors have been identified in the protein databases as homologues of mA3-CDA2, hA3F-CDA1 or hA3B-CDA1 (see Tables 3A, 3B and 3C). Their biological equivalents (e.g., having at least about 80%, 85%, 90%, 95%, 97%, 98%, 99%, 99.5% sequence identity, or having one, two, or three amino acid addition/deletion/substitution, and having nucleobase deaminase inhibitor activity) can also be prepared with known methods in the art, such as conservative amino acid substitutions.

When the nucleobase deaminase inhibitor is included, it is fused to the nucleobase deaminase but can be separated by a protease cleavage site. In some embodiments, the base editing system further includes the protease that is capable of cleaving the protease cleavage site.

The protease cleavage site can be any known protease cleavage site (peptide) for any proteases. Non-limiting examples of proteases include TEV protease, TuMV protease, PPV protease, PVY protease, ZIKV protease and WNV protease. In some embodiments, the protease cleavage site is not one for trypsin, chymotrypsin, or furin. The protein sequences of example proteases and their corresponding cleavage sites are provided in Table 3.

TABLE 3
Example Sequences

| | | SEQ ID |
|---|---|---|
| Name | Sequence | NO: |
| Mouse APOBEC3 | MSSSTLSNICLTKGLPETRFWVEGRRMDPLSEEEFYSQFYNQRVK | 7 |
| cytidine deaminase | HLCYYHRMKPYLCYQLEQFNGQAPLKGCLLSEKGKQHAEILFLD | |
| domain 2 | KIRSMELSQVTITCYLTWSPCPNCAWQLAAFKRDRPDLILHIYTSR | |
| | LYFHWKRPFQKGLCSLWQSGILVDVMDLPQFTDCWTNFVNPKR | |
| | PFWPWKGLEIISRRTQRRLRRIKESWGLQDLVNDFGNLQLGPPMS | |
| Human | MKPHFRNTVERMYRDTFSYNFYNRPILSRRNTVWLCYEVKTKGP | 8 |
| APOBEC3F | SRPRLDAKIFRGQVYSQPEHHAEMCFLSWFCGNQLPAYKCFQIT | |
| cytidine deaminase | WFVSWTPCPDCVAKLAEFLAEHPNVTLTISAARLYYYWERDYRR | |
| domain 1 | ALCRLSQAGARVKIMDDEEFAYCWENFVYSEG | |
| Human | MNPQIRNPMERMYRDTFYDNFENEPILYGRSYTWLCYEVKIKRG | 9 |
| APOBEC3B | RSNLLWDTGVFRGQVYFKPQYHAEMCFLSWFCGNQLPAYKCFQ | |
| cytidine deaminase | ITWFVSWTPCPDCVAKLAEFLSEHPNVTLTISAARLYYYWERDY | |
| domain 1 | RRALCRLSQAGARVKIMDYEEFAYCWENFVYNEGQ | |
| TEV protease N- | MGESLFKGPRDYNPISSTICHLTNESDGHTTSLYGIGFGPFIITNKH | 10 |
| terminal domain | LFRRNNGTLLVQSLHGVFKVKNTTTLQQHLIDGRDMIIIRMPKDF | |
| | PPFPQKLKFREPQREERICLVTTNFQT | |
| TEV protease C- | MKSMSSMVSDTSCTFPSSDGIFWKHWIQTKDGQCGSPLVSTRDG | 11 |
| terminal domain | FIVGIHSASNFTNTNNYFTSVPKNFMELLTNQEAQQWVSGWRLN | |
| | ADSVLWGGHKVFMVKPEEPFQPVKEATQ | |
| TEV protease | ENLYFQS | 12 |
| cleavage site | | |
| TuMV protease | MASSNSMFRGLRDYNPISNNICHLTNVSDGASNSLYGVGFGPLIL | 13 |
| | TNRHLFERNNGELVIKSRHGEFVIKNTTQLHLLPIPDRDLLLIRLPK | |
| | DVPPFPQKLGFRQPEKGERICMVGSNFQTKSITSIVSETSTIMPVEN | |
| | SQFWKHWISTKDGQCGSPMVSTKDGKILGLHSLANFQNSINYFA | |
| | AFPDDFAEKYLHTIEAHEWVKHWKYNTSAISWGSLNIQASQPSG | |
| | LFKVSKLISDLDSTAVYAQ | |
| TuMV protease | GGCSHQS | 14 |
| cleavage site | | |
| PPV protease | MASSKSLFRGLRDYNPIASSICQLNNSSGARQSEMFGLGFGGLIVT | 15 |
| | NQHLFKRNDGELTIRSHHGEFVVKDTKTLKLLPCKGRDIVIIRLPK | |
| | DFPPFPRRLQFRTPTTEDRVCLIGSNFQTKSISSTMSETSATYPVDN | |
| | SHFWKHWISTKDGHCGLPIVSTRDGSILGLHSLANSTNTQNFYAA | |
| | FPDNFETTYLSNQDNDNWIKQWRYNPDEVCWGSLQLKRDIPQSP | |
| | FTICKLLTDLDGEFVYTQ | |
| PPV protease | QVVVHQSK | 16 |
| cleavage site | | |
| PVY protease | MASAKSLMRGLRDFNPIAQTVCRLKVSVEYGASEMYGFGFGAYI | 17 |
| | VANHHLFRSYNGSMEVQSMHGTFRVKNLHSLSVLPIKGRDIILIK | |
| | MPKDFPVFPQKLHFRAPTQNERICLVGTNFQEKYASSIITETSTTY | |
| | NIPGSTFWKHWIETDNGHCGLPVVSTADGCIVGIHSLANNAHTTN | |
| | YYSAFDEDFESKYLRTNEHNEWVKSWVYNPDTVLWGPLKLKDS | |
| | TPKGLFKTTKLVQDLIDHDVVVEQ | |
| PVY protease | YDVRHQSR | 18 |
| cleavage site | | |
| ZIKV protease | MASDMYIERAGDITWEKDAEVTGNSPRLDVALDESGDFSLVEED | 19 |
| | GPPMREGGGGSGGGGSGALWDVPAPKEVKKGETTDGVYRVMT | |
| | RRLLGSTQVGVGVMQEGVFHTMWHVTKGAALRSGEGRLDPYW | |
| | GDVKQDLVSYCGPWKLDAAWDGLSEVQLLAVPPGERARNIQTL | |
| | PGIFKTKDGDIGAVALDYPAGTSGSPILDKCGRVIGLYGNGVVIK | |
| | NGSYVSAITQGKREEETPVECFE | |
| ZIKV protease | KERKRRGA | 20 |
| cleavage site | | |
| WNV protease | MASSTDMWIERTADISWESDAEITGSSERVDVRLDDDGNFQLMN | 21 |
| | DPGAPWKGGGGSGGGGGVLWDTPSPKEYKKGDTTTGVYRIMTR | |
| | GLLGSYQAGAGVMVEGVFHTLWHTTKGAALMSGEGRLDPYWG | |
| | SVKEDRLCYGGPWKLQHKWNGQDEVQMIVVEPGKNVKNVQTK | |
| | PGVFKTPEGEIGAVTLDFPTGTSGSPIVDKNGDVIGLYGNGVIMPN | |
| | GSYISAIVQGERMDEPIPAGFEPEML | |
| WNV protease | KQKKRGGK | 22 |
| cleavage site | | |
| MS2 | ACAUGAGGAUCACCCAUGU | 23 |
| sgRNA scaffold | GUUUGAGAGCUAGGCCA ACAUGAGGAUCACCCAUGU CUGC | 24 |
| with 2 × MS2 | AGGGCCUAGCAAGUUCAAAUAAGGCUAGUCCGUUAUCAACU | |
| | UG GCCAACAUGAGGAUCACCCAUGUCUGCAGGGCC AAGUG | |
| | GCACCGAGUCGGUGC | |
| PP7 | GGAGCAGACGAUAUGGCGUCGCUCC | 25 |
| sgRNA scaffold | GUUUGAGAGCUACCGGAGCAGACGAUAUGGCGUCGCUCCGG | 26 |
| with 2 x PP7 | UAGCAAGUUCAAAUAAGGCUAGUCCGUUAUCAACUUGGAGC | |
| | AGACGAUAUGGCGUCGCUCCAAGUGGCACCGAGUCGGUGC | |
| boxB | GCCCUGAAGAAGGGC | 27 |
| sgRNA scaffold | GUUUGAGAGCUAGGGCCCUGAAGAAGGGCCCUAGCAAGUUC | 28 |
| with 2 x boxB | AAAUAAGGCUAGUCCGUUAUCAACUUGGGCCCUGAAGAAGG | |
| | GCCCAAGUGGCACCGAGUCGGUGC | |
| MS2 coat protein | MASNFTQFVLVDNGGTGDVTVAPSNFANGIAEWISSNSRSQAYK | 29 |
| (MCP) | VTCSVRQSSAQNRKYTIKVEVPKGAWRSYLNMELTIPIFATNSDC | |
| | ELIVKAMQGLLKDGNPIPSAIAANSGIY | |
| PP7 coat protein | MGSKTIVLSVGEATRTLTEIQSTADRQIFEEKVGPLVGRLRLTASL | 30 |
| (PCP) | RQNGAKTAYRVNLKLDQADVVDSGLPKVRYTQVWSHDVTIVA | |
| | NSTEASRKSLYDLTKSLVATSQVEDLVVNLVPLGR | |
| boxB coat protein | MGNARTRRRERRAEKQAQWKAAN | 31 |
| (N22p) | | |
| UGI | TNLSDIIEKETGKQLVIQESILMLPEEVEEVIGNKPESDILVHTAYD | 32 |
| | ESTDENVMLLTSDAPEYKPWALVIQDSNGENKIKML | |
| P2A | GSGATNFSLLKQAGDVEENPGP | 33 |
| T2A | GSGEGRGSLLTCGDVEENPGP | 34 |
| E2A | GSGQCTNYALLKLAGDVESNPGP | 35 |
| IRES | CCCCTCTCCCTCCCCCCCCCCTAACGTTACTGGCCGAAGCCGC | 36 |
| | TTGGAATAAGGCCGGTGTGCGTTTGTCTATATGTTATTTTCCAC | |
| | CATATTGCCGTCTTTTGGCAATGTGAGGGCCCGGAAACCTGGC | |
| | CCTGTCTTCTTGACGAGCATTCCTAGGGGTCTTTCCCCTCTCGC | |
| | CAAAGGAATGCAAGGTCTGTTGAATGTCGTGAAGGAAGCAGT | |
| | TCCTCTGGAAGCTTCTTGAAGACAAACAACGTCTGTAGCGACC | |
| | CTTTGCAGGCAGCGGAACCCCCCACCTGGCGACAGGTGCCTCT | |
| | GCGGCCAAAAGCCACGTGTATAAGATACACCTGCAAAGGCGG | |
| | CACAACCCCAGTGCCACGTTGTGAGTTGGATAGTTGTGGAAAG | |
| | AGTCAAATGGCTCTCCTCAAGCGTATTCAACAAGGGGCTGAA | |
| | GGATGCCCAGAAGGTACCCCATTGTATGGGATCTGATCTGGGG | |
| | CCTCGGTACACATGCTTTACATGTGTTTAGTCGAGGTTAAAAA | |
| | AACGTCTAGGCCCCCCGAACCACGGGGACGTGGTTTTCCTTTG | |
| | AAAAACACGATGATAATATGGCCACAACC | |

TABLE 3A
mA3CDA2 Core Sequence Related Domains

| | | SEQ ID |
|---|---|---|
| Name | Sequence | NO: |
| Mouse APOBEC3 | SEKGKQHAEILFLDKIRSMELSQVTITCYLTWSPCPNCAWQLAAFKRD | 37 |
| cytidine deaminase | RPDLILHIYTSRLYFHWKRPFQKGLC | |
| domain 2 core | | |
| (AA282-AA355) | | |
| Mus spicilegus A3 | SEKGKQHAEILFLDKIRSMELSQVTITCYLTWSPCPNCAWQLAAFKRD | 38 |
| (AA248-AA321) | RPDLIPHIYTSRLYFHWKRPFQKGLC | |
| Cricetulus | SEKGKQHAEILFLDKIRSMELSQVTITCYLTWSPCPNCAWRLAAFKRD | 39 |
| longicaudatus A3 | RPDLILHIYTSRLYFHWKRPFQKGLC | |
| (AA249-AA322) | | |
| Mus terricolor A3 | SEKGKQHAEILFLNKIRSMELSQVTITCYLTWSPCPNCAWQLAAFKKD | 40 |
| (AA248-AA321) | RPDLILHIYTSRLYFHWKRPFQKGLC | |
| Mus caroli A3 | SKKGKQHAEILFLDKIRSMELSQVTITCYLTWSPCPNCAWQLAAFKRD | 41 |
| (AA260-AA333) | HPDLILHIYTSRLYFHWKRPFQKGLC | |
| Mus pahari A3 | SKKGKQHAEILFLEKIRSMELSQMRITCYLTWSPCPNCAWQLAAFQKD | 42 |
| (AA263-AA336) | RPDLILHIYTSRLYFHWRRIFQKGLC | |
| Mus shortridgei A3 | SKKGKQHAEILFLEKIRSMELSQMRITCYLTWSPCPNCAWQLAAFQKD | 43 |
| (AA233-AA306) | RPDLILHIYTSRLYFHWRRIFQKGLC | |
| Mus setulosus A3 | SKKGKQHAEILFLDKIRSMELSQVRITCYLTWSPCPNCAWQLETFKKD | 44 |
| (AA29-AA302) | RPDLILHIYTSRLYFHWKRAFQEGLC | |
| Grammomys | SKKGKPHAEILFLDKMWSMEELSQVRITCYLTWSPCPNCARQLAAFKK | 45 |
| surdaster A3 | DHPGLILRIYTSRLYFYWRRKFQKGLC | |
| (AA270-AA344) | | |
| Rattus norvegicus A3 | KKGEQHVEILFLEKMRSMELSQVRITCYLTWSPCPNCARQLAAFKKDH | 46 |
| (AA256-AA328) | PDLILRIYTSRLYFYWRKKFQKGLC | |
| Mastomys coucha A3 | SKKGRQHAEILFLEKVRSMQLSQVRITCYLTWSPCPNCAWQLAAFKM | 47 |
| (AA258-AA331) | DHPDLILRIYASRLYFHWRRAFQKGLC | |
| Cricetulus griseus | NKKGKHAEILFIDEMRSLELGQVQITCYLTWSPCPNCAQELAAFKSDH | 48 |
| A3B (AA235- | PDLVLRIYTSRLYFHWRRKYQEGLC | |
| AA307) | | |
| Peromyscus leucopus | NKKGKHAEILFIDEMRSLELGQARITCYLTWSPCPNCAQKLAAFKKDH | 49 |
| A3 (AA266-AA338) | PDLVLRVYTSRLYFHWRRKYQEGLC | |
| Mesocricetus auratus | NKKDKHAEILFIDKMRSLELCQVRITCYLTWSPCPNCAQELAAFKKDH | 50 |
| A3 (AA268-AA340) | PDLVLRIYTSRLYFHWRRKYQEGLC | |
| Microtus ochrogaster | NKKGKHAEILFIDEMRSLKLSQERITCYLTWSPCPNCAQELAAFKRDHP | 51 |
| A3B (AA266- | GLVLRIYASRLYFHWRRKYQEGLC | |
| AA338) | | |
| Nannospalax galili | NKRAKHAEILLIDMMRSMELGQVQITCYITWSPCPTCAQELAAFKQDH | 52 |
| A3 (AA231-AA302) | PDLVLRIYASRLYFHWKRKFQKGL | |
| Meriones | NKKGRHAEICLIDEMRSLGLGKAQITCYLTWSPCRKCAQELATFKKDH | 53 |
| unguiculatus A3 | PDLVLRVYASRLYFHWSRKYQQGLC | |
| (AA233-AA305) | | |
| Dipodomys ordii A3 | NKKGHHAEIRFIERIRSMGLDPSQDYQITCYLTWSPCLDCAFKLAKLKK | 54 |
| (AA256-AA330) | DFPRLTLRIFTSRLYFHWIRKFQKGL | |
| Jaculus jaculus A3 | NKKGKHAEARFVDKMRSMQLDHALITCYLTWSPCLDCSQKLAALKR | 55 |
| (AA303-AA374) | DHPGLTLRIFTSRLYFHWVKKFQEGL | |
| Chinchilla lanigera | SPQKGHHAESRFIKRISSMDLDRSRSYQITCFLTWSPCPSCAQELASFKR | 56 |
| A3H (AA86-AA161) | AHPHLRFQIFVSRLYFHWKRSYQAGL | |
| Heterocephalus | KKGYHAESRFIKRICSMDLGQDQSYQVTCFLTWSPCPHCAQELVSFKR | 57 |
| glaber A3 (AA277- | AHPHLRLQIFTARLFFHWKRSYQEGL | |
| AA350) | | |
| Octodon degus A3 | KKGQHAEIRFIERIHSMALDQARSYQITCFLTWSPCPFCAQELASFKSTH | 58 |
| (AA256-AA329) | PRVHLQIFVSRLYFHWKRSYQEGL | |
| Urocitellus parryii | NKKGHHAEIRFIKKIRSLDLDQSQNYEVTCYLTWSPCPDCAQELVALT | 59 |
| A3 (AA256-AA330) | RSHPHVRLRLFTSRLYFHWFWSFQEGL | |
| Aotus nancymaae | NRHAEICFIDEIESMGLDKTQCYEVTCYLTWSPCPSCAQKLAAFTKAQ | 60 |
| A3H (AA75-AA146) | VHLNLRIFASRLYYHWRSSYQKGL | |
| Cebus capucinus | NRHAEICFIDEIESMGLDKTQCYEVTCYLTWSPCPSCAQKLVAFAKAQ | 61 |
| imitator A3H (AA55- | DHLNLRIFASRLYYHWRRRYKEGL | |
| AA126) | | |
| Saimiri boliviensis | HVEICFIDKIASMELDKTQCYDVTCYLTWSPCPSCAQKLAAFAKAQDH | 62 |
| boliviensis A3H | LNLRIFASRLYYHWRRSYQKGL | |
| (AA56-AA125) | | |
| Homo sapiens A3H | NKKKCHAEICFINEIKSMGLDETQCYQVTCYLTWSPCSSCAWELVDFI | 63 |
| (AA49-AA123) | KAHDHLNLGIFASRLYYHWCKPQQKGL | |
| Homo sapiens | ENKKKCHAEICFINEIKSMGLDETQCYQVTCYLTWSPCSSCAWELVDFI | 64 |
| ARP10 (AA48- | KAHDHLNLGIFASRLYYHWCKPQQKGL | |
| AA123) | | |
| Pan paniscus A3H | NKKKCHAEICFINEIKSMGLDETQCYQVTCYLTWSPCSSCAWKLVDFI | 65 |
| (AA49-AA123) | QAHDHLNLRIFASRLYYHWCKPQQEGL | |
| Symphalangus | NKKKRHAEIRFINKIKSMGLDETQCYQVTCYLTWSPCPSCAWELVDFI | 66 |
| syndactylus A3H | KAHDHLNLGIFASRLYYHWCRHQQEGL | |
| (AA49-AA123) | | |
| Macaca mulatta A3H | NKKKDHAEIRFINKIKSMGLDETQCYQVTCYLTWSPCPSCAGELVDFIK | 67 |
| (AA49-AA123) | AHRHLNLRIFASRLYYHWRPNYQEGL | |
| Theropithecus gelada | NKKKEHAEIRFINKIKSMGLDETQCYQVTCYLTWSPCPSCAGKLVDFIK | 68 |
| A3H (AA54-AA128) | AHHHLNLRIFASRLYYHWRPNYQEGL | |
| Mandrillus | NKKKHHAEIHFINKIKSMGLDETQCYQVTCYLTWSPCPSCARELVDFIK | 69 |
| leucophaeus A3H | AHRHLNLRIFASRLYYHWRPHYQEGL | |
| (AA49-AA123) | | |
| Bos grunniens A3 | NKKQRHAEIRFIDKINSLDLNPSQSYKIICYITWSPCPNCANELVNFITRN | 70 |
| (AA74-AA148) | NHLKLEIFASRLYFHWIKPFKMGL | |
| Bubalus bubalis A3 | NKKQRHAEIRFIDKINSLDLNPSQSYKIICYITWSPCPNCASELVDFITRN | 71 |
| (AA74-AA148) | DHLDLQIFASRLYFHWIKPFKRGL | |
| Odocoileus | NKKQRHAEIRFIDKINSLNLDRRQSYKIICYITWSPCPRCASELVDFITGN | 72 |
| virginianus texanus | DHLNLQIFASRLYFHWKKPFQRGL | |
| A3H (AA209- | | |
| AA283) | | |
| Sus scrofa A3 | NKKKRHAEIRFIDKINSLNLDQNQCYRIICYVTWSPCHNCAKELVDFIS | 73 |
| (AA51-AA125) | NRHHLSLQLFASRLYFHWVRCYQRGL | |
| Ceratotherium simum | NKKKRHAEIRFIDKIKSLGLDRVQSYEITCYITWSPCPTCALELVAFTRD | 74 |
| simum A3B (AA232- | YPRLSLQIFASRLYFHWRRRSIQGL | |
| AA306) | | |
| Equus caballus A3H | NKKKRHAEIRFIDKINSLGLDQDQSYEITCYVTWSPCATCACKLIKFTR | 75 |
| (AA79-AA153) | KFPNLSLRIFVSRLYYHWFRQNQQGL | |
| Enhydra lutris | KKKRHAEIRFIDSIRALQLDQSQRFEITCYLTWSPCPTCAKELAMFVQD | 76 |
| kenyoni A3B | HPHISLRLFASRLYFHWRWKYQEGL | |
| (AA243-AA316) | | |
| Leptonychotes | KKKRHAEIRFIDNIKALRLDTSQRFEITCYVTWSPCPTCAKELVAFVRD | 77 |
| weddellii A3H | HRHISLRLFASRLYFHWLRENKKGL | |
| (AA50-AA123) | | |
| Ursus arctos | NKKKRHAEIRFIDKIRSLORDSSQTFEITCYVTWSPCFTCAEELVAFVRD | 78 |
| horribilis A3F | HPHVRLRLFASRLYFHWLRKYQEGL | |
| (AA552-AA626) | | |
| Panthera leo | NKKKRHAEICFIDKIKSLTRDTSQRFEIICYITWSPCPFCAEELVAFVKD | 79 |
| bleyenberghi A3H | NPHLSLRIFASRLYVHWRWKYQQGL | |
| (AA50-AA124) | | |
| Panthera tigris | NKKKRHAEICFIDKIKSLTRDTSQRFEIICYITWSPCPFCAEELVAFVKD | 80 |
| sumatrae A3H | NPHLSLRIFASRLYVHWRWKYQQGL | |
| (AA50-AA124) | | |
| Tupaia belangeri A3 | NKKHRHAEVRFIAKIRSMSLDLDQKHQLTCYLTWSPCPSCAQELVTFM | 81 |
| (AA46-AA120) | AESRHLNLQVFVSRLYFHWQRDFQQGL | |

TABLE 3B
hA3FCDA1 Core Sequence Related Domains

| | | SEQ ID |
|---|---|---|
| Name | Sequence | NO: |
| Pan troglodytes A3F | RRNTVWLCYEVKTKGPSRPRLDTKIFRGQVYFEPQYHAEMCFLSWFC | 82 |
| (AA29-AA136) | GNQLPAYKCFQITWFVSWTPCPDCVAKLAEFLAEHPNVTLTISAARLY | |
| | YYWERDYRRALCR | |
| Pan paniscus A3F | RRNTVWLCYEVKTKGPSRPRLDTKIFRGQVYFQFENHAEMCFLSWFC | 83 |
| (AA29-AA136) | GNQLPAYKCFQITWFVSWTPCPDCVAKLAEFLAEHPNVTLTISAARLY | |
| | YYWERDYRRALCR | |
| Colobus angolensis | RRNTVWLCYEVKTRGPSMPTWGAKIFRGQVYFEPQYHAEMCFLSWFC | 84 |
| palliatus A3F | GNQLPAYKCFQITWFVSWTPCPDCVGKVAEFLAEHPNVTLTISAARLY | |
| (AA29-AA136) | YYWETDYRRALCR | |
| Macaca mulatta A3F | RRNTVWLCYEVKTRGPSMPTWDTKIFRGQVYSKPEHHAEMCFLSRFC | 85 |
| (AA29-AA136) | GNQLPAYKRFQITWFVSWTPCPDCVAKVAEFLAEHPNVTLTISAARLY | |
| | YYWETDYRRALCR | |
| Macaca fascicularis | RRNTVWLCYEVKTRGPSVPTWGTKIFRGQVYSKPEHHAEMCFLSWFC | 86 |
| A3F | GNQLPTYKRFQITWFVSWTPCPDCVAKVAEFLAEHPNVTLTISAARLY | |
| (AA29-AA136) | YYWETDYRRALCR | |
| Rhinopithecus | RRNTVWLCYEVKTRGPSMPTWGAKIFRGQVYFEPQYHAEMCFLSWFC | 87 |
| roxellana A3F | GNQLPAYKRFQITWFVSWTPCPDCVAKVAEFLAEHPNVTLTISAARLY | |
| (AA29-AA136) | YYWETDYRRALCR | |
| Rhinopithecus bieti | RRNTVWLCYEVKTRGPSMPTWGAKIFRGQVYFEPQYHAEMCFLSWFC | 88 |
| A3F | GNQLPAYKRFQITWFVSWTPCPDCVAKVAEFLAEHPNVTLTISAARLY | |
| (AA18-AA125) | YYWETDYRRALCR | |
| Rhinopithecus | RRNTVWLCYEVKTRGPSMPTWGAKIFRGQVYFEPQYHAEMCFLSWFC | 89 |
| roxellana A3F | GNQLPAYKRFQITWFVSWTPCPDCVAKVAEFLAEHPNVTLTISAARLY | |
| (AA29-AA136) | YYWETDYRRALCR | |
| Macaca mulatta A3F | RRNTVWLCYEVKTRGPSMPTWDTKIFRGQVYSKPEHHAEMCFLSRFC | 90 |
| (AA40-AA147) | GNQLPAYKRFQITWFVSWTPCTDCVAKVAEFLAEHPNVTLTISAARLY | |
| | YYWETDYRRALCR | |
| Trachypithecus | RRNTVWLCYEVKTRGPSMPTWGAKIFRGQVYFEPQYHAEMCFLSWFC | 91 |
| francoisi A3F | GNQLPAYKRFRITWFVSWTPCPDCVAKVAEFLAEHPNVTLTISAARLY | |
| (AA40-AA147) | YYWETDYRRALCR | |
| Gorilla gorilla A3F | RRNTVWLCYEVKTKGPSRPPLDAKIFRGQVYFEPQYHAEMCFLSWFC | 92 |
| (AA29-AA127) | GNQLPAYKCFQITWFVSWTPCPDCVAKLAEFLAEHPNVTLTISAARLY | |
| | YYWE | |
| Papio anubis A3F | RRNTVWLCYEVKTRGPSMPTWDAKIFRGQVYFQPQYHAEMCFLSRFC | 93 |
| (AA29-AA136) | GNQLPAYKRFQITWFVSWTPCPDCVVKVTEFLAEHPNVTLTISAARLY | |
| | YYWETDYRRALCR | |
| Pongo abelii A3F | RRNTVWLCYKVKTKGPSRPPLNAKIFRGQVYFEPQYHAEMCFLSWFC | 94 |
| (AA29-AA136) | GNQLSAYERFQITWFVSWTPCPDCVAMLAEFLAEHPNVTLTVSAARL | |
| | YYYWERDYRGALRR | |
| Macaca leonina A3F | RRNTVWLCYEVKTRGPSMPTWGTKIFRGQVCFEPQYHAEMCFLSRFC | 95 |
| (AA29-AA136) | GNQLPAYKRFQITWFVSWTPCPDCVAKVAEFLAEHPNVTLTISAARLY | |
| | YYWETDYRRALCR | |
| Macaca nemestrina | RRNTVWLCYEVKTRGPSMPTWGTKIFRGQVCFEPQYHAEMCFLSRFC | 96 |
| A3F | GNQLPAYKRFQITWFVSWTPCPDCVAKVAEFLAEHPNVTLTISAARLY | |
| (AA29-AA136) | YYWETDYRRALCR | |
| Homo sapiens A3B | RSYTWLCYEVKIKRGRSNLLWDTGVFRGQVYFEPQYHAEMCFLSWFC | 97 |
| (AA30-AA137) | GNQLPAYKCFQITWFVSWTPCPDCVAKLAEFLSEHPNVTLTISAARLY | |
| | YYWERDYRRALCR | |
| Gorilla gorilla gorilla | RSYNWLCYEVKIKRGRSNLLWNTGVFRGQMYSQPEHHAEMCFLSWF | 98 |
| A3B | CGNQLPAYKCFQITWFVSWTPCPDCVAKLAEFLAEYPNVTLTISAARL | |
| (AA30-AA137) | YYYWERDYRRALCR | |
| Pan troglodytes A3B | RSYTWLCYEVKIRRGHSNLLWDTGVFRGQMYSQPEHHAEMCFLSWF | 99 |
| (AA30-AA137) | CGNQLSAYKCFQITWFVSWTPCPDCVAKLAKFLAEHPNVTLTISAARL | |
| | YYYWERDYRRALCR | |
| Theropithecus gelada | RRNTVWLCYEVKTRGPSMPTWGTKIFRGQVYFQPQYHAEMCFLSRFC | 100 |
| A3F | GNQLPAYKRFQITWFVSWNPCPDCVAKVIEFLAEHPNVTLTISAARLY | |
| (AA29-AA136) | YYWGRDWRRALRR | |
| Mandrillus | RRNTVWLCYKVKTRGPSMPTWGTKIFRGQVYFQPQYHAEMCFLSWF | 101 |
| leucophaeus A3F | CGNQLPAYKRFQITWFVSWTPCPDCVVKVAEFLAEHPNVTLTISAARL | |
| (AA29-AA130) | YYYWETDY | |
| Gorilla gorilla gorilla | RSYTWLCYEVKIKRGRSNLLWDTGVFRGQMYSQPEHHAEMCFLSWF | 102 |
| A3B | CGNQLPAYKCFQITWFVSWTPCLDCVAKLAEFLAEYPNVTLTISTARL | |
| (AA30-AA137) | YYYWERDYRRALCR | |
| Pan paniscus A3B | RSYTWLCYEVKIRRGHSNLLWDTGVFRGQMYSQPEHHAEMYFLSWF | 103 |
| (AA30-AA137) | CGNQLSAYKCFQITWFVSWTPCPDCVAKLAEFLAEHPNVTLTISAARL | |
| | YYYWERDYRRALCR | |
| Hylobates moloch | RSYTWLCYEVKIRKDPSKLPWDTGVFRGQMYFQPEYHAEMCFLSWFC | 104 |
| A3B | GNQLPAYKRFQITWFVSWTPCPDCVAKVAEFLAEHPNVTLTISAARLY | |
| (AA30-AA137) | YYWEKDWQRALCR | |
| Symphalangus | RRNTVWLCYEVKTKDPSRPRLDTKIFRGKVYFQLENHAEMCFLSWFC | 105 |
| syndactylus A3G | GNQLPANRCFQITWFVSWNPCLPCVAKVTKFLAEHPNVTLTISAARLY | |
| (AA22-AA129) | YYRARDWRRALRR | |
| Macaca mulatta A3B | RSYTWLCYEVKIRKDPSKLPWDTGVFRGQMYSKPEHHAEMCFLSWFC | 106 |
| (AA30-AA137) | GNQLPAHKRFQITWFVSWTPCPDCVAKVAEFLAEYPNVTLTISAARLY | |
| | YYWETDYRRALCR | |
| Chlorocebus sabaeus | RSYTWLCYEVKIRKDPSKLPWDTGVFRGQMYSKPEHHAEMCFLSWFC | 107 |
| A3B | GNQLPAHKRFQITWFVSWTPCPDCVAKVAEFLAEYPNVTLTISAARLY | |
| (AA30-AA137) | YYWETDYRRALCR | |
| Nomascus | RRSYTWLCYEVKIRKDPSKLPWDTGVFRGQMYFQPEYHAEMCFLSWF | 108 |
| leucogenys A3B | CGNQLPAYKRFQITWFVSWTPCPDCVAKVAVFLAEHPNVTLTISAARL | |
| (AA30-AA137) | YYYWEKDWQRALCR | |
| Trachypithecus | RSYTWLCYEVKIRKDPSKLPWDTGVFRGQVYSEPEHHAEMYFLSWFC | 109 |
| francoisi A3B | GNQLPAYKRFWITWFVSWTPCPDCVAKLAEFLTEHPNVTLTISAARLY | |
| (AA30-AA137) | YYRGRDWRRALCR | |
| Trachypithecus | RSYTWLCYEVKKRKDPSKLPWDTGVFRGQVYSEPEHHAEMYFLSWF | 110 |
| francoisi A3F | CGNQLPAYKRFWITWFVSWTPCPDCVAKVAEFLAEHPKVTLTISAARL | |
| (AA22-AA129) | YYYWDRDWRRALCR | |
| Rhinopithecus bieti | RSYTWLCYEVKIRKDPSKLPWDTGVFRGQVYSEPEHHAEMYFLSWFC | 111 |
| A3F | GNQLPAYKRFQITWFVSWTPCPDCVAKVAEFLTEHPNVTLTISAARLY | |
| (AA30-AA137) | YYRGRDWRRALCR | |
| Rhinopithecus | RSYTWLCYEVKIRKDPSKLPWDTGVFRGQVYSEPEHHAEMYFLSWFC | 112 |
| roxellana A3B | GNQLPAYKRFQITWFVSWTPCPDCVAKVAEFLTEHPNVTLTISAARLY | |
| (AA30-AA137) | YYRGRDWRRALCR | |
| Pongo abelii A3F | RRNYTWLCYEVKIRKDPSKLAWDTGVFRGQVYSQPEHHAEMCFLSW | 113 |
| (AA29-AA128) | FCGNQLSAYERFQITWFVSWTPCPDCVAKLAEFLAEHPNVTLTVSAAR | |
| | LYYYWE | |
| Macaca mulatta A3B | RSYTWLCYEVKIRKDPSKLPWDTGVFRGQVYSKPEHHAEMCFLSRFC | 114 |
| (AA30-AA137) | GNQLPAYKRFQITWFVSWNPCPDCVAKVIEFLAEHPNVTLTISTARLY | |
| | YYWGRDWQRALCR | |
| Macaca leonina A3B | RSYTWLCYEVKIRKDPSKLPWDTGVFRGQVYSKPEHHAEMCFLSRFC | 115 |
| (AA30-AA137) | GNQLPAYKRFQITWFVSWNPCPDCVVKVIEFLAEHPNVTLTISTARLY | |
| | YYWGRDWQRALCR | |
| Macaca nemestrina | RSYTWLCYEVKIRKDPSKLPWDTGVFRGQVYSKPEHHAEMCFLSRFC | 116 |
| A3B | GNQLPAYKRFQITWFVSWNPCPDCVAKVTEFLAEHPNVTLTISTARLY | |
| (AA30-AA137) | YYWGRDWQRALCR | |
| Macaca mulatta A3D | RSYTWLCYEVKIRKDPSKLPWDTGVFRGQVYFQPQYHAEMCFLSWFC | 117 |
| (AA30-AA137) | GNQLPAYKRFQITWFVSWNPCPDCVAKVTEFLAEHPNVTLTISVARLY | |
| | YYRGKDWRRALCR | |
| Pongo abelii A3F | RRNYTWLCYEVKIRKDPSKLAWDTGVFRGQVLPKLQSNHRREVYFEP | 118 |
| (AA29-AA149) | QYHAEMCFLSWFCGNQLSAYERFQITWFVSWTPCPDCVAMLAEFLAE | |
| | HPNVTLTVSAARLYYYWERDYRGALRR | |
| Erythrocebus patas | RRYTWLCYEVKIKKDPSKLPWDTGVFQGQVRPKFQSNRRYEVYFQPQ | 119 |
| A3DE | YHAEMCFLSWFCGNQLPAYKHFQITWFVSWNPCPDCVAKVTEFLAEH | |
| (AA30-AA149) | PNVTLTISAARLYYYWGKDWRRALCR | |
| Pan troglodytes A3B | MYSQPEHHAEMCFLSWFCGNQLSAYKCFQITWFVSWTPCPDCVAKLA | 120 |
| (AA1-AA79) | KFLAEHPNVTLTISAARLYYYWERDYRRALCR | |
| Macaca mulatta | RSYTWLCYEVKIRKDPSKLPWDTGVFRGQVRPKLQSNRRYEVYFQPQ | 121 |
| A3DE | YHAEMCFLSWFCGNQLPAYKRFQITWFVSWNPCPDCVAKVTEFLAEH | |
| (AA30-AA149) | PNVTLTISAARLYYYWGKDWRRALRR | |
| Piliocolobus | RRYTWLCYEVKIMKDHSKLPWYTGVFRGQVYFEPQNHAEMCFLSWF | 122 |
| tephrosceles A3F | CGNQLPAYECCQITWFVSWTPCPDCVAKVTEFLAEHPNVTLTISAARL | |
| (AA30-AA137) | YYYRGRDWRRALRR | |
| Macaca leonina A3D | RSYTWLCYEVKIRKDPSKLPWYTGVFRGQVYFQPQYHAEMCFLSWFC | 123 |
| (AA30-AA137) | GNQLPANKRFQITWFVSWNPCPDCVAKVTEFLAEHPNVTLTISVARLY | |
| | YYRGKDWRRALRR | |
| Macaca nemestrina | RSYTWLCYEVKIRKDPSKLPWDTGVFRDQVYFQPQYHAEMCFLSWFC | 124 |
| A3D | GNQLPANKRFQITWFVSWNPCPDCVTKVTEFLAEHPNVTLTISVARLY | |
| (AA30-AA137) | YYRGKDWRRALRR | |
| Chlorocebus aethiops | RRYTWLCYEVKIKKDPSKLPWDTGVFPGQVRPKFQSNRRYEVYFQPQ | 125 |
| A3DE | YHAEMYFLSWFCGNQLPAYKHFQITWFVSWNPCPDCVAKVTEFLAEH | |
| (AA30-AA149) | RNVTLTISAARLYYYWGKDWRRALCR | |
| Macaca mulatta | RSYTWLCYEVKIRKDPSKLPWDTGVFRGQVRPKLQSNRRYELSNWEC | 126 |
| A3D | RKHVYFQPQYHAEMCFLSWFCGNQLPANKRFQITWFVSWNPCPDCVA | |
| (AA30-AA158) | KVTEFLAEHPNVTLTISAARLYYYWGKDWRRALRR | |

TABLE 3C
hA3BCDA1-Related Domains

| | | SEQ ID |
|---|---|---|
| Name | Sequence | NO: |
| Gorilla A3B (AA29- | GRSYNWLCYEVKIKRGRSNLLWNTGVFRGQMYSQPEHHAEMCFLSW | 127 |
| AA138) | FCGNQLPAYKCFQITWFVSWTPCPDCVAKLAEFLAEYPNVTLTISTARL | |
| | YYYWERDYRRALCRL | |
| Pan paniscus A3B | GRSYTWLCYEVKIRRGHSNLLWDTGVFRGQMYSQPEHHAEMYFLSW | 128 |
| (AA29-AA138) | FCGNQLPAYKCFQITWFVSWTPCPDCVAKLAEFLAEHPNVTLTISAAR | |
| | LYYYWERDYRRALCRL | |
| Pan troglodytes A3B | GRSYTWLCYEVKIRRGHSNLLWDTGVFRGQMYSQPEHHAEMCFLSW | 129 |
| (AA29-AA138) | FCGNQLSAYKCFQITWFVSWTPCPDCVAKLAKFLAEHPNVTLTISAAR | |
| | LYYYWERDYRRALCRL | |
| Gorilla A3F (AA30- | RNTVWLCYEVKTKGPSRPPLDAKIFRGQVYFEPQYHAEMCFLSWFCG | 130 |
| AA137) | NQLPAYKCFQITWFVSWTPCPDCVAKLAEFLAEHPNVTLTISAARLYY | |
| | YWE | |
| Pan troglodytes A3F | RNTVWLCYEVKTKGPSRPRLDTKIFRGQVYFEPQYHAEMCFLSWFCG | 131 |
| (AA30-AA137) | NQLPAYKCFQITWFVSWTPCPDCVAKLAEFLAEHPNVTLTISAARLYY | |
| | YWERDYRRALCRL | |
| Human sapiens A3F | RNTVWLCYEVKTKGPSRPRLDAKIFRGQVYSQPEHHAEMCFLSWFCG | 132 |
| (AA30-AA137) | NQLPAYKCFQITWFVSWTPCPDCVAKLAEFLAEHPNVTLTISAARLYY | |
| | YWERDYRRALCRL | |
| Macaca leonine | RNTVWLCYEVKTRGPSMPTWGTKIFRGQVCFEPQYHAEMCFLSRFCG | 133 |
| A3F (AA30-AA137) | NQLPAYKRFQITWFVSWTPCPDCVAKVAEFLAEHPNVTLTISAARLYY | |
| | YWETDYRRALCRL | |
| Macaca nemestrina | RNTVWLCYEVKTRGPSMPTWGTKIFRGQVCFEPQYHAEMCFLSRFCG | 134 |
| A3F (AA30-AA137) | NQLPAYKRFQITWFVSWTPCPDCVAKVAEFLAEHPNVTLTISAARLYY | |
| | YWETDYRRALCRL | |
| Rhinopithecus | RNTVWLCYEVKTRGPSMPTWGAKIFRGQVYFEPQYHAEMCFLSWFC | 135 |
| roxellana A3F | GNQLPAYKRFQITWFVSWTPCPDCVAKVAEFLAEHPNVTLTISAARLY | |
| (AA30-AA137) | YYWETDYRRALCRL | |
| Mandrillus | RNTVWLCYKVKTRGPSMPTWGTKIFRGQVYFQPQYHAEMCFLSWFC | 136 |
| leucophaeus A3F | GNQLPAYKRFQITWFVSWTPCPDCVVKVAEFLAEHPNVTLTISAARLY | |
| (AA30-AA130) | YYWETDY | |
| Macaca mulatta A3F | RNTVWLCYEVKTRGPSMPTWDTKIFRGQVYSKPEHHAEMCFLSRFCG | 137 |
| (AA30-AA137) | NQLPAYKRFQITWFVSWTPCPDCVAKVAEFLAEHPNVTLTISAARLYY | |
| | YWETDYRRALCRL | |
| Theropithecus gelada | RNTVWLCYEVKTRGPSMPTWGTKIFRGQVYFQPQYHAEMCFLSRFCG | 138 |
| A3F (AA30-AA137) | NQLPAYKRFQITWFVSWNPCPDCVAKVIEFLAEHPNVTLTISAARLYY | |
| | YWGRDWRRALRRL | |
| Cercocebus atys A3B | GRSYTWLCYEVKIRKDPSKLPWYTGVFRGQVYSKPEHHAEMCFLSRF | 139 |
| (AA29-AA138) | CGNQLPAYKRFQITWFVSWNPCPDCVAKVIEFLAEHPNVTLTISAARL | |
| | YYYWSRDWQRALCRL | |
| Macaca fascicularis | GRSYTWLCYEVKIRKDPSKLPWDTGVFRGQVYSKPEHHAEMCFLSRF | 140 |
| A3B (AA29-AA138) | CGNQLPAYKRFQITWFVSWNPCPDCVAKVIEFLAEHPNVTLTISTARLY | |
| | YYWGRDWQRALCRL | |
| Macaca mulatta A3B | GRSYTWLCYEVKIRKDPSKLPWDTGVFRGQVYSKPEHHAEMCFLSRF | 141 |
| (AA29-AA138) | CGNQLPAYKRFQITWFVSWNPCPDCVAKVIEFLAEHPNVTLTISTARLY | |
| | YYWGRDWQRALCRL | |
| Macaca leonina A3B | GRSYTWLCYEVKIRKDPSKLPWDTGVFRGQVYSKPEHHAEMCFLSRF | 142 |
| (AA29-AA138) | CGNQLPAYKRFQITWFVSWNPCPDCVVKVIEFLAEHPNVTLTISTARLY | |
| | YYWGRDWQRALCRL | |
| Mandrillus | GRSYTWLCYEVKIRKDPSKLPWYTGVFRGQVYSKPEHHAEMCFLSRF | 143 |
| leucophaeus A3B | CGNQLPAYKRFQITWFVSWNPCPDCVAKVIEFLAEHPNVTLTIFTARLY | |
| (AA29-AA138) | YYWGRDWQRALCRL | |
| Macaca nemestrina | GRSYTWLCYEVKIRKDPSKLPWDTGVFRGQVYSKPEHHAEMCFLSRF | 144 |
| A3B (AA29-AA138) | CGNQLPAYKRFQITWFVSWNPCPDCVAKVTEFLAEHPNVTLTISTARL | |
| | YYYWGRDWQRALCRL | |
| Rhinopithecus bieti | GRSYTWLCYEVKIRKDPSKLPWDTGVFRGQVYSEPEHHAEMYFLSWF | 145 |
| A3F (AA29-AA138) | CGNQLPAYKRFQITWFVSWTPCPDCVAKVAEFLTEHPNVTLTISAARL | |
| | YYYRGRDWRRALCRL | |
| Rhinopithecus | GRSYTWLCYEVKIRKDPSKLPWDTGVFRGQVYSEPEHHAEMYFLSWF | 146 |
| roxellana A3B | CGNQLPAYKRFQITWFVSWTPCPDCVAKVAEFLTEHPNVTLTISAARL | |
| (AA29-AA138) | YYYRGRDWRRALCRL | |
| Chlorocebus sabaeus | GRSYTWLCYEVKIRKDPSKLPWDTGVFRGQMYSKPEHHAEMCFLSWF | 147 |
| A3B (AA29-AA138) | CGNQLPAHKRFQITWFVSWTPCPDCVAKVAEFLAEYPNVTLTISAARL | |
| | YYYWETDYRRALCRL | |
| Nomascus | RSYTWLCYEVKIRKDPSKLPWDTGVFRGQMYFQPEYHAEMCFLSWFC | 148 |
| leucogenys A3B | GNQLPAYKRFQITWFVSWTPCPDCVAKVAVFLAEHPNVTLTISAARLY | |
| (AA30-AA138) | YYWEKDWQRALCRL | |
| Cercocebus atys A3F | GRSYTWLCYEVKIKKYPSKLLWDTGVFQGQVYFQPQYHAEMCFLSRF | 149 |
| (AA29-AA138) | CGNQLPAYKRFQITWFVSWNPCPDCVAKVTEFLAEHPNVTLTISAARL | |
| | YYYWEKDXRRALRRL | |
| Papio anubis A3F | GRSYTWLCYEVKIKEDPSKLLWDTGVFQGQVYFQPQYHAEMCFLSRF | 150 |
| (AA29-AA138) | CGNQLPAYKRFQITWFVSWNPCPDCVAKVTEFLAEHPNVTLTISAARL | |
| | YYYWGRDWRRALRRL | |
| Chlorocebus aethiops | GRRYTWLCYEVKIKKDPSKLPWDTGVFPGQVRPKFQSNRRYEVYFQP | 151 |
| A3D (AA29-AA150) | QYHAEMYFLSWFCGNQLPAYKHFQITWFVSWNPCPDCVAKVTEFLAE | |
| | HRNVTLTISAARLYYYWGKDWRRALCRL | |
| Chlorocebus sabaeus | GRRYTWLCYEVKIKKDPSKLPWDTGVFPGQPQYHAEMYFLSWFCGN | 152 |
| A3D (AA29-AA134) | QLPAYKHFQITWFVSWNPCPDCVAKVTEFLAEHRNVTLTISAARLYYY | |
| | WGKDWRRALCRL | |
| Chlorocebus sabaeus | GRRYTWLCYEVKIKKDPSKLPWDTGVFPGQVRPKFQSNRRQKVYFQP | 153 |
| A3F (AA29-AA150) | QYHAEMYFLSWFCGNQLPAYKHFQITWFVSWNPCPDCVAKVTEFLAE | |
| | HRNVTLTISAARLYYYWGKDWRRALCRL | |
| Erythrocebus patas | GRRYTWLCYEVKIKKDPSKLPWDTGVFQGQVRPKFQSNRRYEVYFQP | 154 |
| A3D (AA29-AA150) | QYHAEMCFLSWFCGNQLPAYKHFQITWFVSWNPCPDCVAKVTEFLAE | |
| | HPNVTLTISAARLYYYWGKDWRRALCRL | |
| Macaca fascicularis | GRSYTWLCYEVKIRKDPSKLPWDTGVFRGQVRPKLQSNRRYELSNWE | 155 |
| A3D (AA29-AA159) | CRKRVYFQPQYHAEMYFLSWFCGNQLPANKRFQITWFASWNPCPDCV | |
| | AKVTEFLAEHPNVTLTISVARLYYYRGKDWRRALRRL | |
| Macaca fascicularis | GRSYTWLCYEVKIRKDPSKLPWDTGVFRGQVYFQPQYHAEMYFLSWF | 156 |
| A3F (AA29-AA138) | CGNQLPANKRFQITWFASWNPCPDCVAKVTEFLAEHPNVTLTISVARL | |
| | YYYRGKDWRRALRRL | |
| Macaca nemestrina | GRSYTWLCYEVKIRKDPSKLPWDTGVFRDQVYFQPQYHAEMCFLSWF | 157 |
| A3D (AA29-AA138) | CGNQLPANKRFQITWFVSWNPCPDCVTKVTEFLAEHPNVTLTISVARL | |
| | YYYRGKDWRRALRRL | |
| Macaca leonina A3D | GRSYTWLCYEVKIRKDPSKLPWYTGVFRGQVYFQPQYHAEMCFLSWF | 158 |
| (AA29-AA138) | CGNQLPANKRFQITWFVSWNPCPDCVAKVTEFLAEHPNVTLTISVARL | |
| | YYYRGKDWRRALRRL | |
| Macaca mulatta A3D | GRSYTWLCYEVKIRKDPSKLPWDTGVFRGQVYFQPQYHAEMCFLSWF | 159 |
| (AA29-AA138) | CGNQLPAYKRFQITWFVSWNPCPDCVAKVTEFLAEHPNVTLTISVARL | |
| | YYYRGKDWRRALCRL | |
| Gorilla A3D (AA29- | GRSYTWLCYEVKIRRGSSNLLWNTGVFRGPVPPKLQSNHRQEVYFQFE | 160 |
| AA150) | NHAEMCFLSWFCGNRLPANRRFQITWFVSWNPCLPCVVKVTKFLAEH | |
| | PNVTLTISAARLYYYRDREWRRVLRRL | |
| Pan paniscus A3D | GRSYTWLCYEVKIKRGCSNLIWDTGVFRGPVLPKLQSNHRQEVYFQFE | 161 |
| (AA29-AA150) | NHAEMCFFSWFCGNRLPANRRFQITWFVSWNPCLPCVVKVTKFLAEH | |
| | PNVTLTISAARLYYYQDREWRRVLRRL | |
| Pan troglodytes A3D | GRSYTWLCYEVKIKRGCSNLIWDTGVFRGPVLPKLQSNHRQEVYFQFE | 162 |
| (AA29-AA150) | NHAEMCFFSWFCGNRLPANRRFQITWFVSWNPCLPCVVKVTKFLAEH | |
| | PNVTLTISAARLYYYQDREWRRVLRRL | |
| Homo sapiens A3D | GRSYTWLCYEVKIKRGRSNLLWDTGVFRGPVLPKRQSNHRQEVYFRF | 163 |
| (AA29-AA150) | ENHAEMCFLSWFCGNRLPANRRFQITWFVSWNPCLPCVVKVTKFLAE | |
| | HPNVTLTISAARLYYYRDRDWRWVLLRL | |
| Nomascus | GRSYTWLCYEVKIRKDPSKLPWDKGVFRGQVLPKFQSNHRQEVYFQL | 164 |
| leucogenys A3D | ENHAEMCFLSWFCGNQLPANRRFQITWFVSWNPCLPCVAKVTEFLAE | |
| (AA29-AA150) | HPNVTLTISAARLYYYRGRDWRRALRRL | |
| Saimiri boliviensis | GKKYTWLCYEVKIKKDTSKLPWNTGVFRGQVNFNPEHHAEMYFLSW | 165 |
| A3C (AA29-AA138) | FRGKLLPACKRSQITWFVSWNPCLYCVAKVAEFLAEHPNVTLTVSTAR | |
| | LYCYWKKDWRRALRKL | |
| Saimiri boliviensis | GKKYTWLCYEVKIKKDTSKLPWNTGVFRGQVNFNPEHHAEMYFLSW | 166 |
| A3F (AA29-AA138) | FRGKLLPACKRSQITWFVSWNPCLYCVAKVAEFLAEHPNVTLTVSTAR | |
| | LYCYWKKDWRRALRKL | |
| Piliocolobus | GRRYTWLCYEVKIMKDHSKLPWYTGVFRGQVYFEPQNHAEMCFLSW | 167 |
| tephrosceles A3F | FCGNQLPAYECCQITWFVSWTPCPDCVAKVTEFLAEHPNVTLTISAAR | |
| (AA36-AA145) | LYYYRGRDWRRALRRL | |
| Colobus angolensis | GRRYTWLCYEVKISKDPSKLPWDTGIFRGQVYFEPQYHAEMCFLSWY | 168 |
| palliatus A3F (AA29- | CGNQLPAYKCFQITWFVSWTPCPDCVGKVAEFLAEHPNVTLTISAARL | |
| AA138) | YYYWETDYRRALCRL | |
| Pongo abelii A3F | RNYTWLCYEVKIRKDPSKLAWDTGVFRGQVLPKLQSNHRREVYFEPQ | 169 |
| (AA30-AA150) | YHAEMCFLSWFCGNQLSAYERFQITWFVSWTPCPDCVAMLAEFLAEH | |
| | PNVTLTVSAARLYYYWERDYRGALRRL | |

In some embodiments, the protease cleavage site is a self-cleaving peptide, such as the 2A peptides. “2A peptides” are 18-22 amino-acid-long viral oligopeptides that mediate “cleavage” of polypeptides during translation in eukaryotic cells. The designation “2A” refers to a specific region of the viral genome and different viral 2As have generally been named after the virus they were derived from. The first discovered 2A was F2A (foot-and-mouth disease virus), after which E2A (equine rhinitis A virus), P2A (porcine teschovirus-1 2A), and T2A (thosea asigna virus 2A) were also identified. A few non-limiting examples of 2A peptides are provided in SEQ ID NO: 33-35.

In some embodiments, the protease cleavage site is a cleavage site (e.g., SEQ ID NO: 12) for the TEV protease. In some embodiments, the TEV protease provided in the base editing system includes two separate fragments, each of which on its own is not active. However, in the presence of the remaining fragment of the TEV protease, they will be able to execute the cleavage. Such an arrangement provides additional control and flexible of the base editing capabilities. The TEV fragments may be the TEV N-terminal domain (e.g., SEQ ID NO: 10) or the TEV C-terminal domain (e.g., SEQ ID NO: 11).

In some embodiments, a fusion protein is provided that includes a mutant mA3CDA1 domain (optionally with a deaminase inhibitor) and a Cas protein.

The term “Cas protein” or “clustered regularly interspaced short palindromic repeats (CRISPR)-associated (Cas) protein” refers to RNA-guided DNA endonuclease enzymes associated with the CRISPR (Clustered Regularly Interspaced Short Palindromic Repeats) adaptive immunity system in Streptococcus pyogenes, as well as other bacteria. Cas proteins include Cas9 proteins, Cas12a (Cpf1) proteins, Cas12b (formerly known as C2c1) proteins, Cas13 proteins and various engineered counterparts. Example Cas proteins include SpCas9, FnCas9, St1Cas9, St3Cas9, NmCas9, SaCas9, AsCpf1, LbCpf1, FnCpf1, VQR SpCas9, EQR SpCas9, VRER SpCas9, SpCas9-NG, xSpCas9, RHA FnCas9, KKH SaCas9, NmeCas9, StCas9, CjCas9, AsCpf1, FnCpf1, SsCpf1, PcCpf1, BpCpf1, CmtCpf1, LiCpf1, PmCpf1, Pb3310Cpf1, Pb4417Cpf1, BsCpf1, EeCpf1, BhCas12b, AkCas12b, EbCas12b, LsCas12b, RfCas13d, LwaCas13a, PspCas13b, PguCas13b, and RanCas13b.

In some embodiments, a peptide linker is optionally provided between each of the fragments in the fusion protein. In some embodiments, the peptide linker has from 1 to 100 amino acid residues (or 3-20, 4-15, without limitation). In some embodiments, at least 10%, 20%, 30%, 40%, 50%, 60%, 70%, 80% or 90% of the amino acid residues of peptide linker are amino acid residues selected from the group consisting of alanine, glycine, cysteine, and serine.

Improved base editing systems are also provided. In one embodiment, the base editing system includes (a) a first fusion protein comprising a nucleobase deaminase (e.g., a mutant mA3CDA1), a nucleobase deaminase inhibitor (e.g., mA3CDA2), and a first RNA recognition peptide (e.g., MCP), wherein the nucleobase deaminase and the nucleobase deaminase inhibitor is separated by a protease cleavage site (e.g., TEV site) that can be cleaved by a protease (e.g., TEV); (b) a second fusion protein comprising an inactive portion of the protease (e.g., TEVc) fused to a second RNA recognition peptide (e.g., N22p) that is different from the first RNA recognition peptide; (c) a second portion of the protease (e.g., TEVn) which, in combination with the first portion, can carry out the protease activity to cleave the protease cleavage site; (d) a helper sgRNA further comprising a first RNA recognition site (e.g., MS2) recognizable by first RNA recognition peptide; (e) a sgRNA further comprising a second RNA recognition site (e.g., boxB) recognizable by the second RNA recognition peptide; and (f) a Cas protein. In some embodiments, the nucleobase deaminase is a mutant protein of the present disclosure.

In some embodiments, the first fusion protein further includes one, two or three uracil glycosylase inhibitor (UGI). In some embodiments, the Cas protein further includes one, two, or three UGI, wherein the UGIs can be cleaved from the Cas protein to become standalone UGI (e.g., each being separate).

Also provided are polynucleotides encoding part of the base editing systems. In one embodiment, a polynucleotide is provided that includes a first fragment encoding (a) a first fusion protein comprising a nucleobase deaminase, a nucleobase deaminase inhibitor, and a first RNA recognition peptide, wherein the nucleobase deaminase and the nucleobase deaminase inhibitor is separated by a protease cleavage site that can be cleaved by a protease; a second fragment encoding (b) a second fusion protein comprising an inactive portion of the protease fused to a second RNA recognition peptide that is different from the first RNA recognition peptide; a third fragment encoding (c) a second portion of the protease which, in combination with the first portion, can carry out the protease activity to cleave the protease cleavage site; a fourth fragment encoding (d) a helper sgRNA further comprising a first RNA recognition site recognizable by first RNA recognition peptide; and a fifth fragment encoding (e) a sgRNA further comprising a second RNA recognition site recognizable by the second RNA recognition peptide, wherein the first, second, and third fragments are configured to be transcribed into a single mRNA molecule.

In some embodiments, the first and second fragments are separated by a first separating sequence encoding a first internal ribosome entry site (IRES, e.g., SEQ ID NO: 36), and the second and third fragments are separated by a second separating sequence encoding a first self-cleavage peptide. Alternatively, in some embodiments, the first and second fragments are separated by a first separating sequence encoding a second self-cleavage peptide, and the second and third fragments are separated by a second separating sequence encoding a second internal ribosome entry site (IRES, e.g., SEQ ID NO: 36). In some embodiments, the nucleobase deaminase is a mutant protein of the present disclosure.

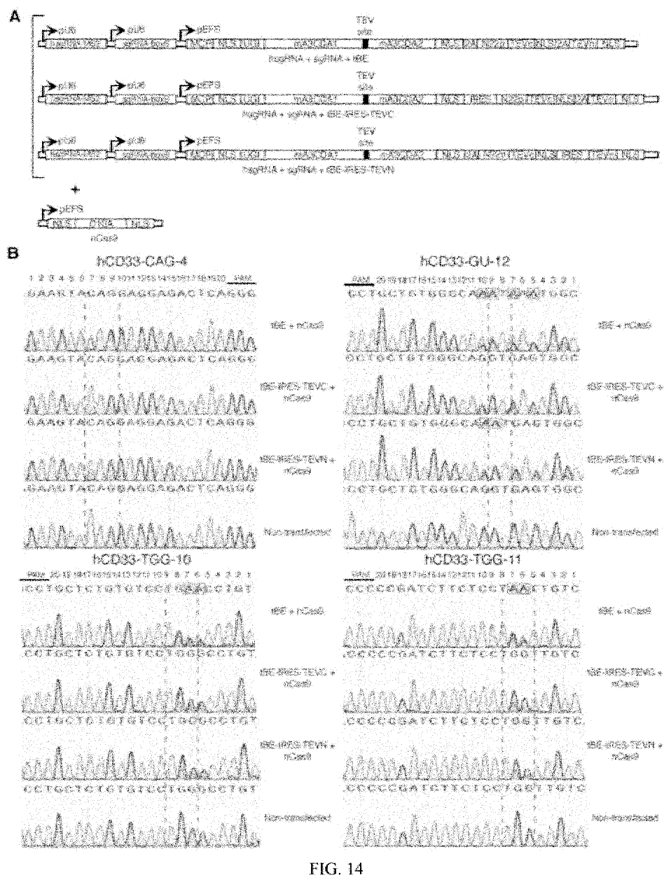

In some embodiments, as illustrated in FIG. 14, each of the fourth fragment and the fifth fragment are regulated and/or transcribed separately from one another. In some embodiments, a further polynucleotide is provided that encodes a Cas protein. In some embodiments, the Cas protein is fused to one or more UGI sequences.

For any fusion protein of the present disclosure, biological equivalents thereof are also provided. In some embodiments, the biological equivalents have at least about 70%, 75%, 80%, 85%, 90%, 95%, 98%, or 99% sequence identity with the reference fusion protein. Preferably, the biological equivalents retained the desired activity of the reference fusion protein. In some embodiments, the biological equivalents are derived by including one, two, three, four, five or more amino acid additions, deletions, substitutions, of the combinations thereof. In some embodiments, the substitution is a conservative amino acid substitution.

A “conservative amino acid substitution” is one in which the amino acid residue is replaced with an amino acid residue having a similar side chain. Families of amino acid residues having similar side chains have been defined in the art, including basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Thus, a nonessential amino acid residue in an immunoglobulin polypeptide is preferably replaced with another amino acid residue from the same side chain family. In another embodiment, a string of amino acids can be replaced with a structurally similar string that differs in order and/or composition of side chain family members.

A base editor that incorporates such a fusion protein has reduced or even no editing capability and accordingly will generate reduced or no off-target mutations. Upon cleavage of the protease cleavage site and release of the nucleobase deaminase inhibitor from the fusion protein at a target site, the base editor that is at the target site will then be able to edit the target site efficiently.

An example base editor is the tBE, which employs a dual sgRNA system, in which a helper sgRNA (hsgRNA) is used to target a site proximate the main target site. In a tBE, the nucleobase deaminase inhibitor is only released when both sgRNA are bound to the target sequences, ensuring that the nucleobase deaminase does not edit at off-target sites.

The first molecule can include just a Cas protein, which has a suitable size for packaging in a common vehicle, AAV. The second molecule includes, among others, a nucleobase deaminase (e.g., a mutant mA3CDA1), a nucleobase deaminase inhibitor (e.g., mA3CDA2), and an RNA recognition peptide (e.g., MCP). A protease cleavage site (e.g., TEV site) is inserted between the nucleobase deaminase and the nucleobase deaminase inhibitor, which enables removal of the nucleobase deaminase inhibitor at proper timing/location. Optionally, the second molecule further includes a UGI.

The third molecule is a fusion between an inactive portion of the protease (e.g., TEVc) fused to a different RNA recognition peptide (e.g., N22p). The fourth molecule is a standalone TEVn which, in combination with the first portion, can carry out the protease activity to remove the nucleobase deaminase inhibitor from the second molecule.

The fifth molecule is a helper sgRNA containing an RNA recognition site (e.g., MS2) recognizable by the RNA recognition peptide in the 2^{nd} molecule. The sixth molecule is a regular sgRNA that contains an RNA recognition site (e.g., boxB) recognizable by the RNA recognition peptide in the 3^{rd} molecule.

At the correct target site in the genome (or RNA), both the hsgRNA and the sgRNA will bind, and each recruits a Cas protein to the binding site. The hsgRNA will also recruit the 2^{nd} molecule by virtue of the MS2-MCP binding, and the sgRNA will recruit the 3^{rd} molecule by virtue of the boxB-N22p binding. Therefore, the TEVc of the 3^{rd} molecule is in contact with the TEV site. Since the standalone TEVn is present in the entire cell, it can also be present here, which ensures that the TEVc is active and cleaves the nucleobase deaminase inhibitor from the nucleobase deaminase in molecule 2, thereby activating the nucleobase deaminase.

In some embodiments, the one or more proteins can be encoded by a single mRNA or construct, while being separated by a sequence encoding a 2A peptide (e.g., SEQ ID NO: 33, 34 or 35) or an internal ribosome entry site (IRES) (e.g., SEQ ID NO: 36). In some embodiments, one or more (e.g., 1, 2, or 3) free UGI sequences are produced from the molecules.

In some embodiments, the distance between the hsgRNA binding site and the regular sgRNA binding site is from 34-91 bp (from PAM to PAM), with the hsgRNA on the upstream.

Also provided, in one embodiment, is a dual guide RNA system. In some embodiments, the system includes a target single guide RNA comprising a first spacer having sequence complementarity to a target nucleic acid sequence proximate to a first PAM site, a helper single guide RNA comprising a second spacer having sequence complementarity to a second nucleic acid sequence proximate to a second PAM site, a clustered regularly interspaced short palindromic repeats (CRISPR)-associated (Cas) protein, and a mutant mA3CDA1 (or a corresponding fusion protein as disclosed herein).

In some embodiments, the second PAM site is located within 150 bases, or alternatively within 140, 130, 120, 110, 100, 95, 94, 93, 92, 91, 90, 89, 88, 87, 86, 85, 84, 83, 82, 81, 80, 75 or 70 bases from the second PAM site. In some embodiments, the second PAM site is located at least 10 bases, or alternatively at least 15, 20, 25, 30, 31, 32, 33, 34, 35, 36, 37, 38, 39, 40, 45, 50, 55, or 60 bases from the first PAM. In some embodiments, the second PAM site is upstream from the first PAM site. In some embodiments, the second PAM site is downstream from the first PAM site. In some embodiments, the distance is from 20-100, 25-95, 30-95, 34-95, 34-91, 34-90, 35-90, 40-90, 40-84, 45-85, or 50-80 bases, without limitation.

In some embodiments, the second (helper) spacer is 8-15 bases in length. In some embodiments, the second spacer is 8-14, 8-13, 8-12, 8-11, 8-10, 9-15, 9-14, 9-13, 9-12, 9-11, 9-10, 10-15, 10-14, 10-13, 10-12, 10-11, 11-15, 11-14, 11-13, 11-12, 12-15, 12-14, 12-13, or 13-15 bases in length. The first spacer, by contrast, is at least 16, 17, 18, or 19 bases in length.

### Methods

The base editors and base editing methods described in this disclosure can be applied to perform high-specificity and high-efficiency base editing in the genome of various eukaryotes.

In one embodiment, the present disclosure provides a method for introducing a C-to-T substitution at a cytosine in a target nucleic acid. In some embodiments, the method entails contacting the target nucleic acid with a CRISPR-associated (Cas) protein, a mutant mA3CDA1 as disclosed herein (or a corresponding fusion protein as disclosed herein), a single-guide RNA (sgRNA), and a helper single-guide RNA (hsgRNA), wherein the sgRNA and the hsgRNA can hybridize to the target nucleic acid.

In some embodiments, the mutant mA3CDA1 has a Y35D or Y35E mutation. In some embodiments, the sgRNA is designed such that the cytosine is between nucleotide positions 6 and 8 3′ to a protospacer adjacent motif (PAM) sequence on the target nucleic acid sequence.

In some embodiments, the mutant mA3CDA1 has K40H and W102Y mutations. In some embodiments, the sgRNA is designed such that the cytosine is between nucleotide positions 4 and 8 3′ to a protospacer adjacent motif (PAM) sequence on the target nucleic acid sequence.

The contacting between the fusion protein (and the guide RNA) and the target polynucleotide can be in vitro, in particular in a cell culture. When the contacting is ex vivo, or in vivo, the fusion proteins can exhibit clinical/therapeutic significance. The in vivo contacting may be administration to a live subject, such as a human, an animal, a yeast, a plant, a bacterium, a virus, without limitation.

### EXAMPLES

### Example 1. Mutant mA3 Catalytic Domain

The instant inventors have developed a new base editing system, transformer base editor (tBE), which can specifically edit cytosine in target regions with no observable off-target mutations. The tBE system is composed of a cytidine deaminase inhibitor (dCDI) and split-TEV system. tBE remains inactive at off-target sites with a cleavable fusion of dCDI domain, thus eliminating unintended mutations. Only when binding at on-target sites, is tBE transformed to cleave off the dCDI domain and catalyzes targeted deamination for precise editing. More specifically, tBE uses a sgRNA (normally 20 nt) to bind at the target genomic site and a helper sgRNA (hsgRNA, normally 10 or 20 nt) to bind at a nearby region upstream to the target genomic site. The binding of two sgRNAs can guide the components of the tBE system to correctly assemble at the target genomic site for base editing.

After analyzing the editable positions at different target sites, we confirmed that the editing window of tBE spans from position 3 to 9 (counting the protospacer adjacent motif (PAM) distal position in target site as 1). It is desirable to develop new tBEs having narrower editing windows.

Through structural research and computer modeling, we selected residues Y35, K37, R39, K40, N66, W102 and Y132 in the catalytic domain of mA3CDA1 for mutations. In the first round, single mutations were tested, including Y35D, K37D, R39A, K40A, N66G, W102Y, W102F and Y132F.

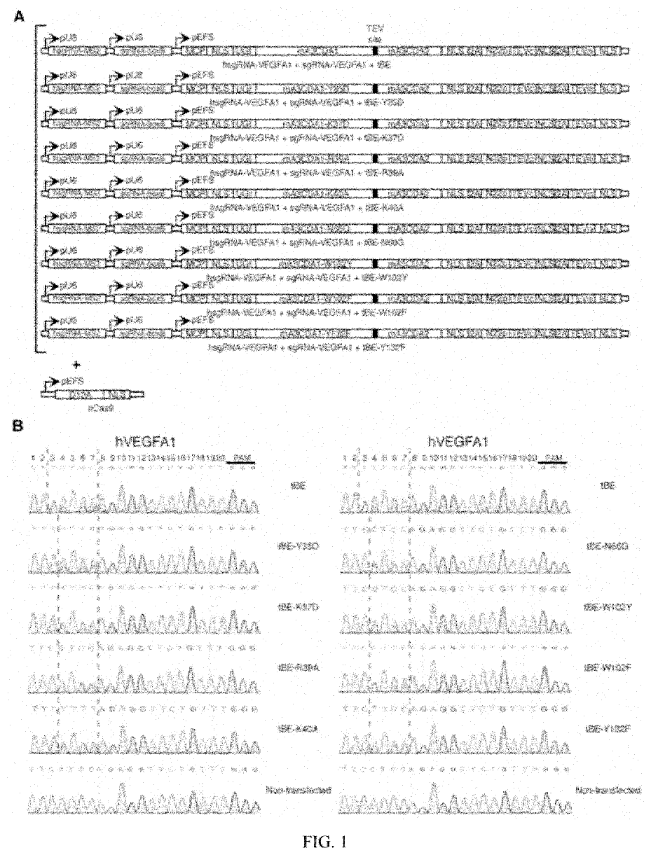

The mutant mA3CDA1 was introduced into the tBE (mA3CDA1+mA3CDA2) system. The resulting base editors tBE-Y35D, tBE-K37D, tBE-R39A, tBE-K40A, tBE-N66G, tBE-W102Y, tBE-W102F and tBE-Y132F were tested with sgRNA and hsgRNA targeting the human VEGFA1 gene. As shown in FIG. 1, these single residue substitutions in the mA3CDA1 region narrowed the editing window, thus improved the editing precision of tBE.

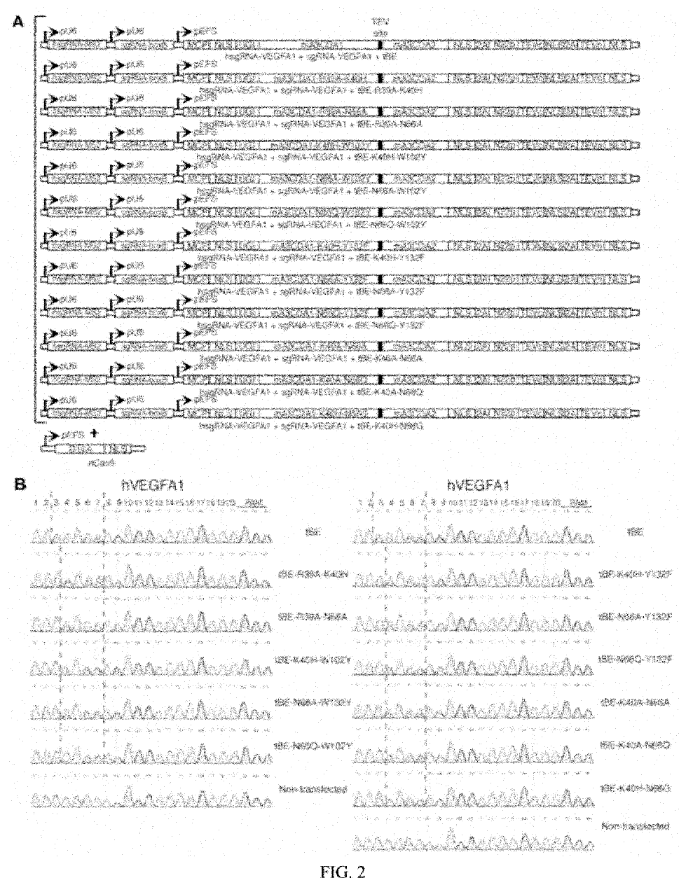

Dual mutations of mA3CDA1 were also tested, including R39A-K40H, R39A-N66A, K40H-W102Y, N66A-W102Y, N66Q-W102Y, K40H-Y132F, N66A-Y132F, N66Q-Y132F, K40A-N66A, K40A-N66Q and K40H-N66G. The resulting base editors tBE-R39A-K40H, tBE-R39A-N66A, tBE-K40H-W102Y, tBE-N66A-W102Y, tBE-N66Q-W102Y, tBE-K40H-Y132F, tBE-N66A-Y132F, tBE-N66Q-Y132F, tBE-K40A-N66A, tBE-K40A-N66Q and tBE-K40H-N66G were also tested on the human VEGFA1 gene. As shown in FIG. 2, these dual-mutant mA3CDA1 also led to narrowed editing windows, thus improving the editing precision of tBE.

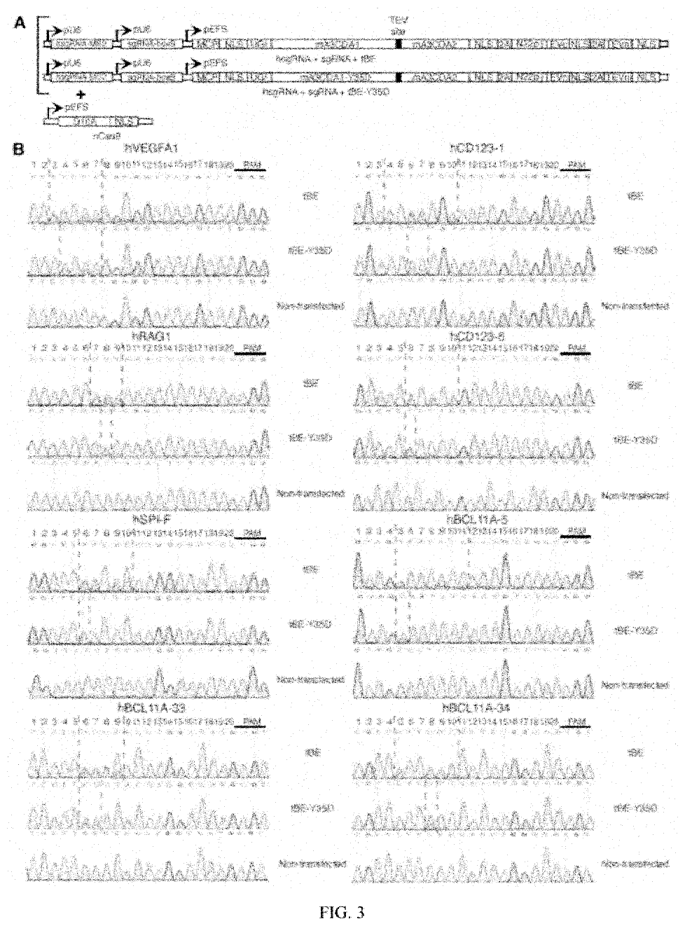

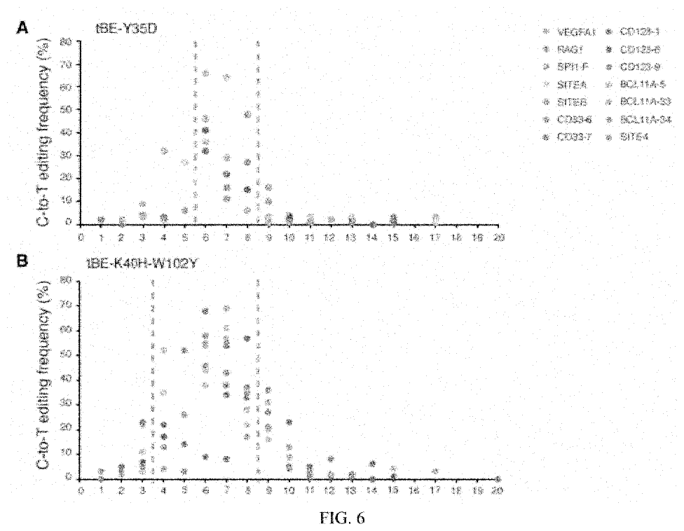

Base editor tBE-Y35D appeared to have the narrowest editing window with high editing efficiency. It was therefore further tested with more target sites, including CD123, RAG1, SPI, and BCL11A. As shown in the results of FIG. 3, the editing window of tBE-Y35D spanned from position 6 to 8 (FIGS. 3 and 6(A)), which is smaller than that of the original tBE (from position 3 to 9).

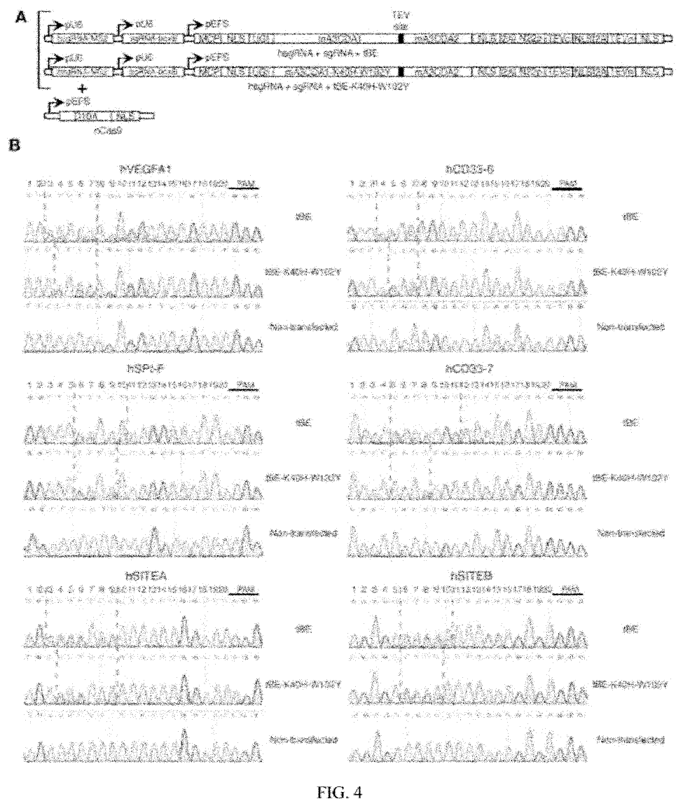

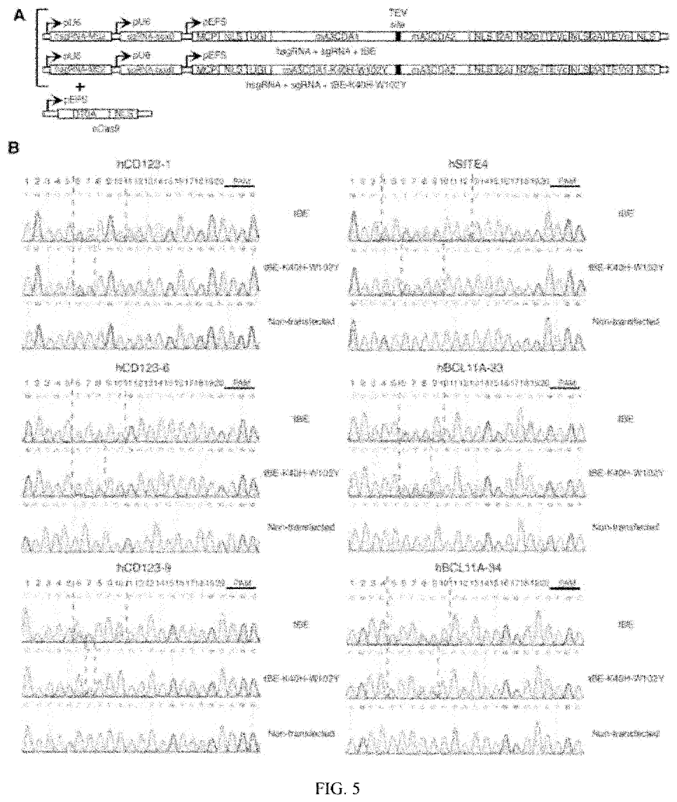

Among the tBE variants with dual mutations, tBE-K40H-W102Y has the narrowest editing window while maintains high editing efficiency. After analyzing the editable positions at more target sites, we found that the editing window of tBE-K40H-W102Y spanned from position 4 to 8 (FIGS. 4, 5 and 6(B)), which is smaller than that of the original tBE (from position 3 to 9).

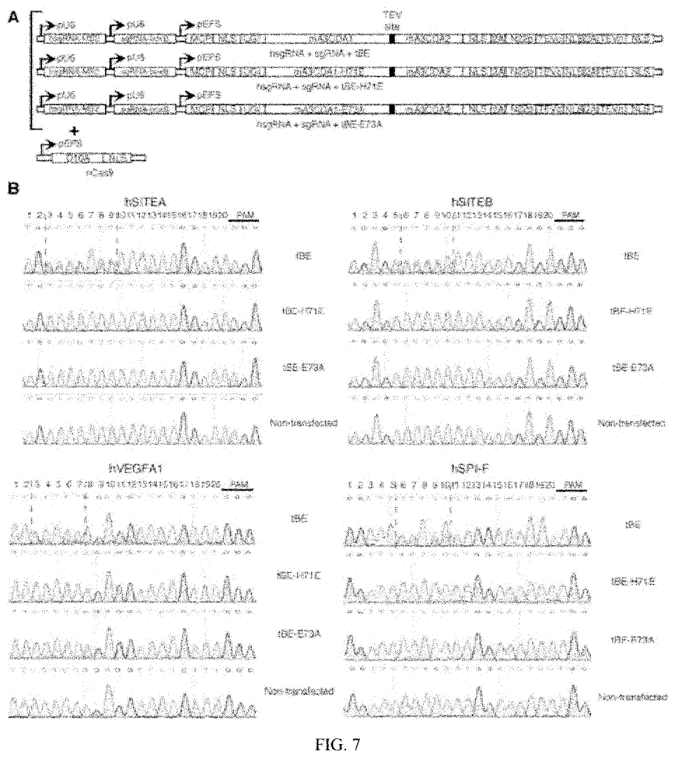

This example further tested which residues are important for retaining the catalytic activity of the mA3CDA1 protein. Through screening, it was determined that residues H71 and E73 are important residues. As shown in FIG. 7, The editing efficiencies of mutants tBE-H71E and tBE-E73A were almost eradicated at the target sites, which shows that H71 and E73 are necessary for the catalytic activity of mA3CDA1.

### Example 2. Further Improved Base Editors with Fused and/or Standalone UGI

To further improve the editing efficiency and fidelity of tBE-mediated base editing, more copies of uracil glycosylase inhibitor (UGIs) proteins are added into tBE system.

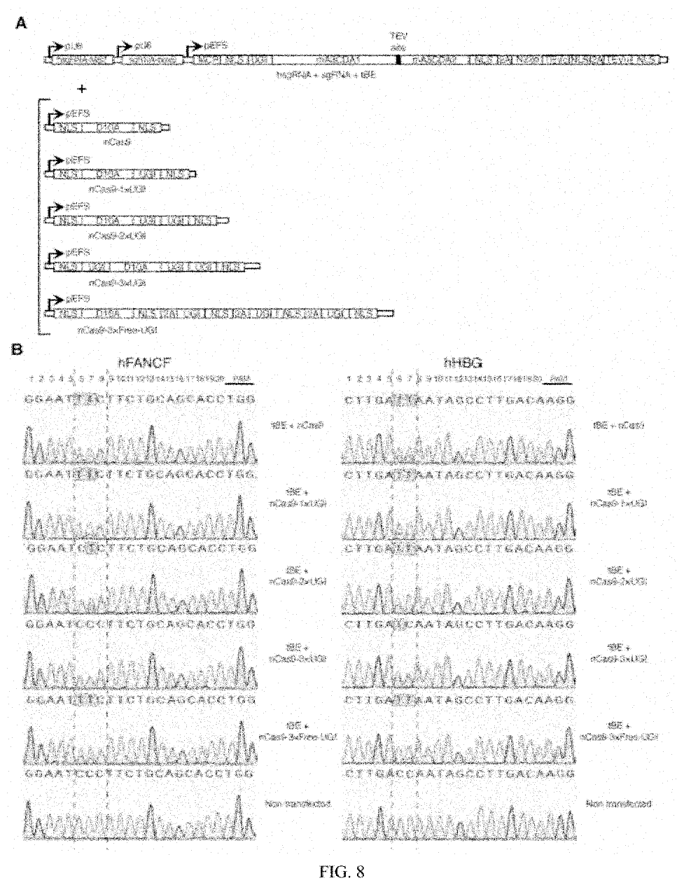

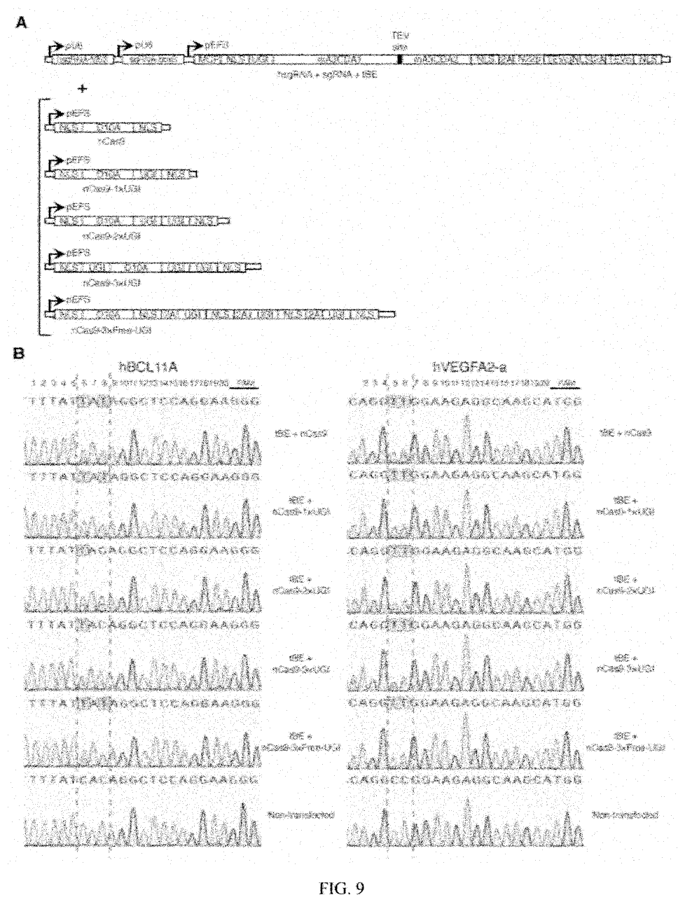

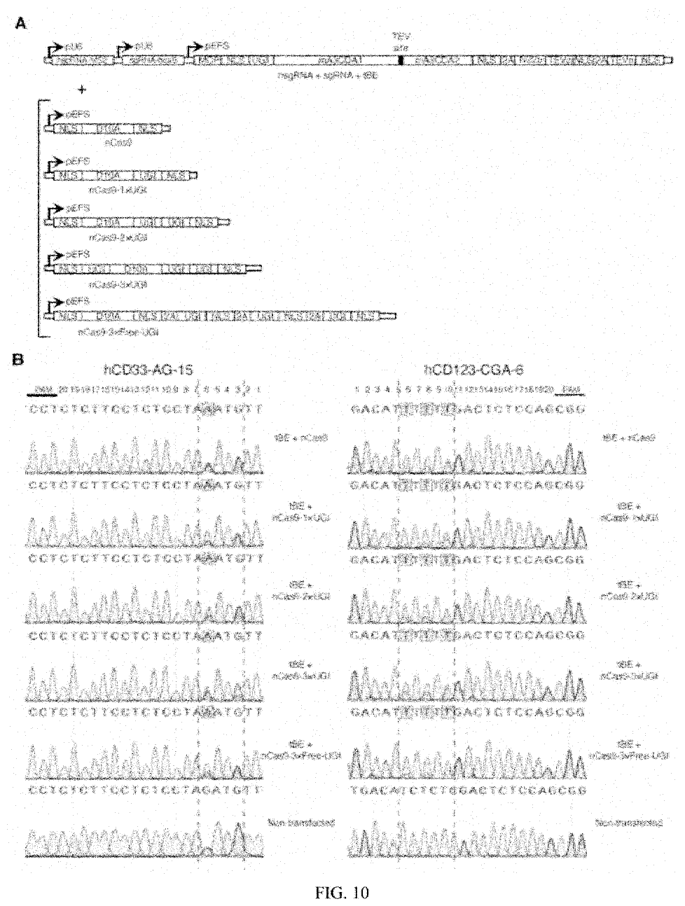

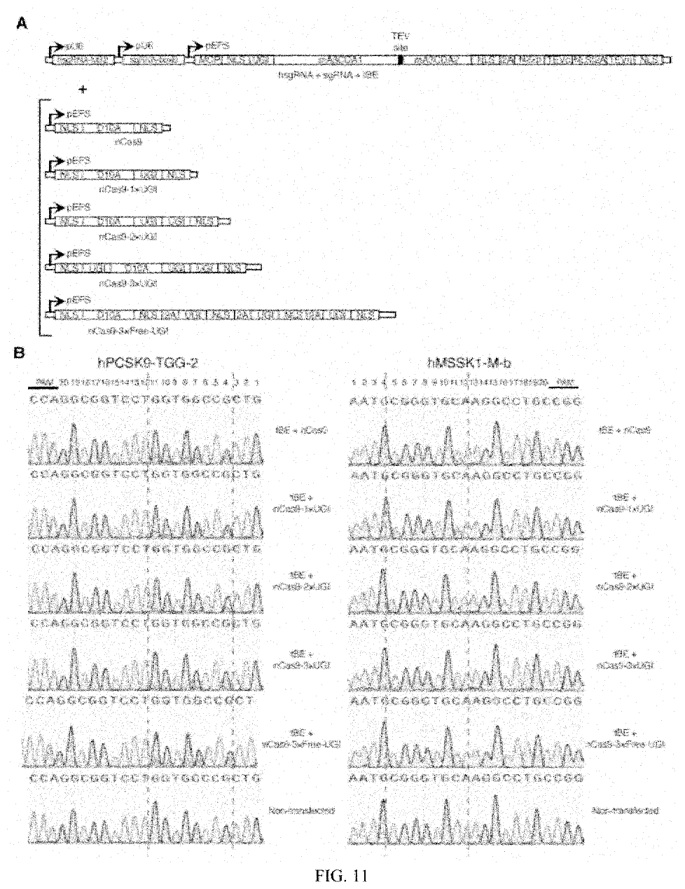

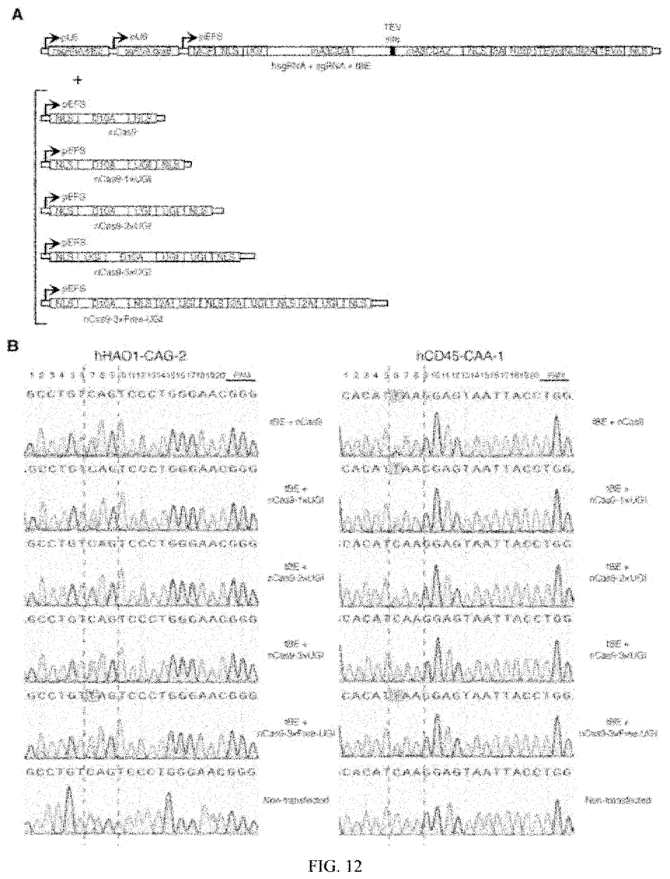

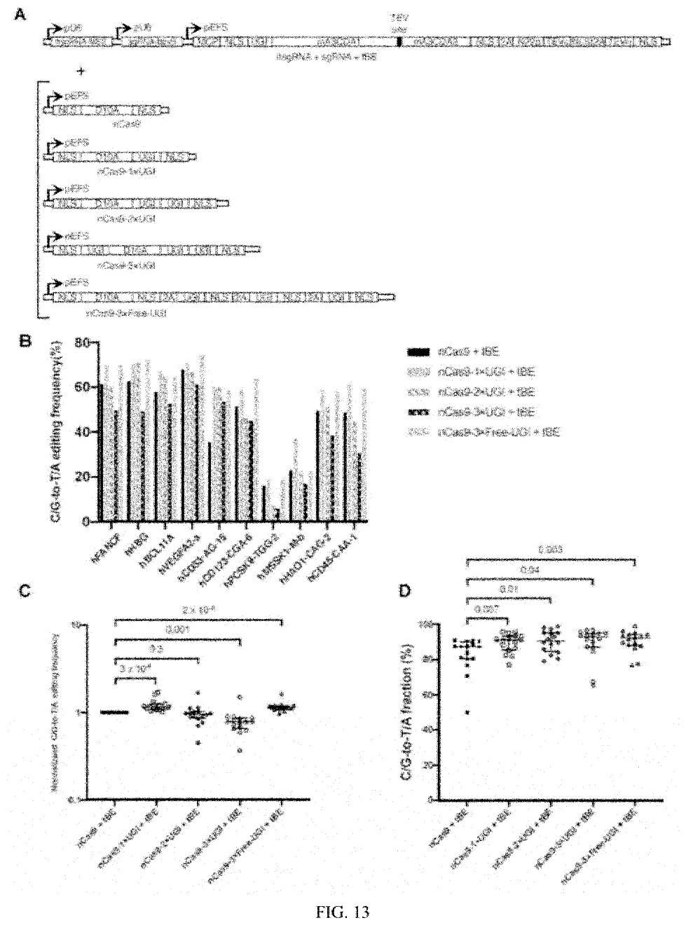

The original tBE vector further co-transfected with different types of nCas9-UGI showed higher C-to-T editing efficiency and fidelity, especially nCas9-1×UGI and nCas9-3×Free-UGI (FIGS. 8-13). We found that nCas9-1×UGI and nCas9-3×Free-UGI suppressed the generation of C-to-A/C-to-G substitutions and simultaneously increasing the desired C-to-T editing (FIGS. 13(B-D)). It is worth noting that both the nCas9-fused UGI type and nCas9-free UGI type could improve the fidelity and efficiency of tBE system.

### Example 3. Further Improved Base Editors with IRES

In the original tBE system, deaminase and split TEV proteases are separated by two 2A peptides to co-express three ORFs under the control of a single promoter.

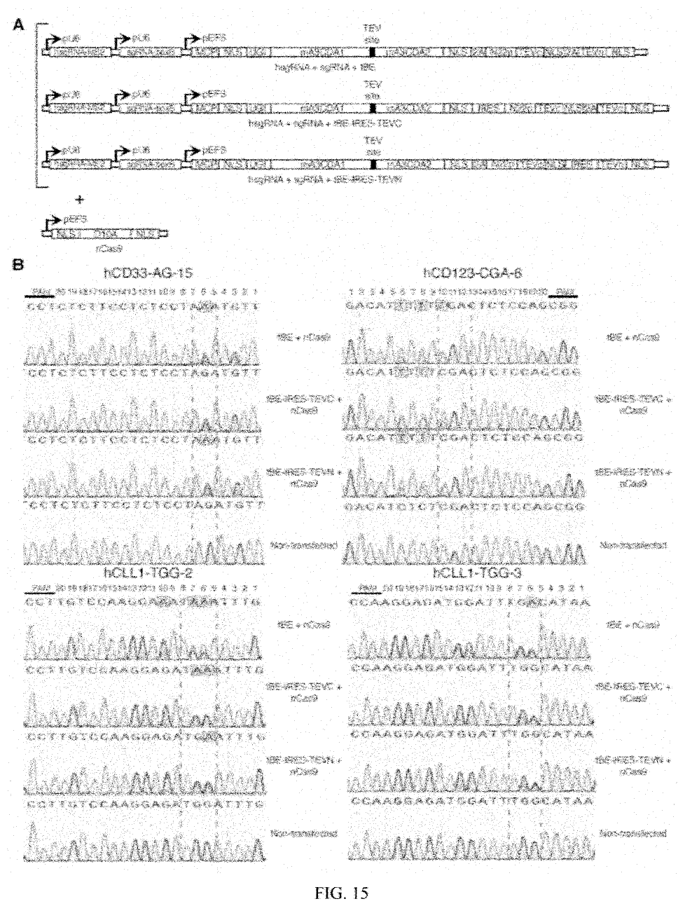

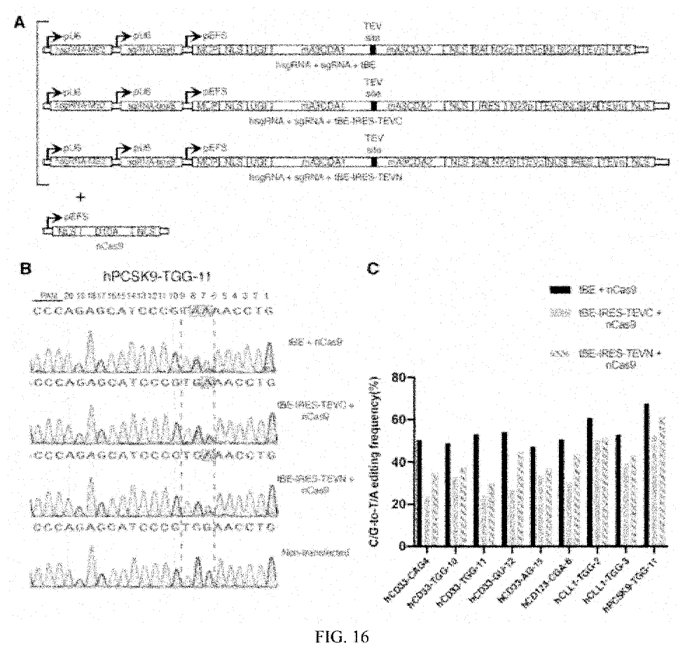

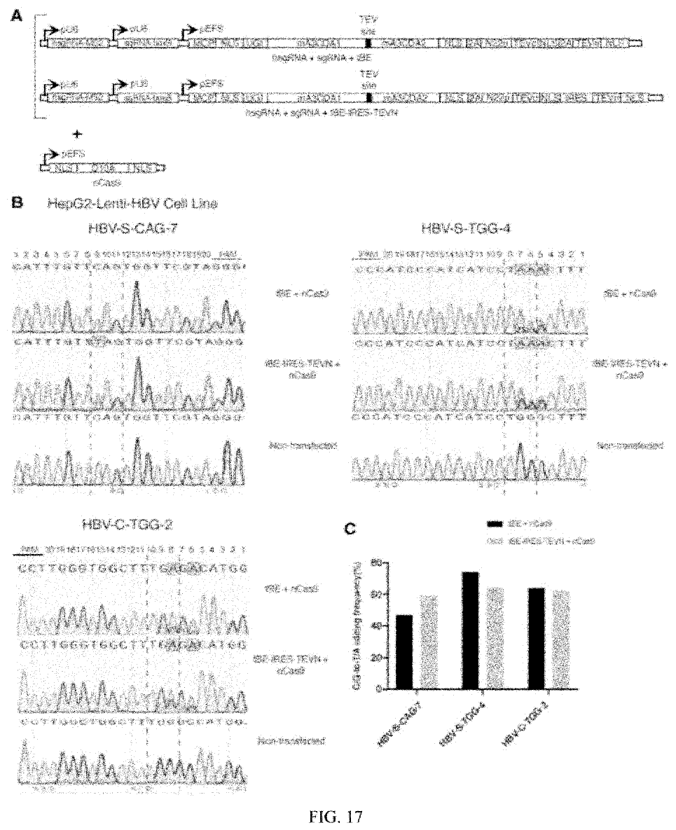

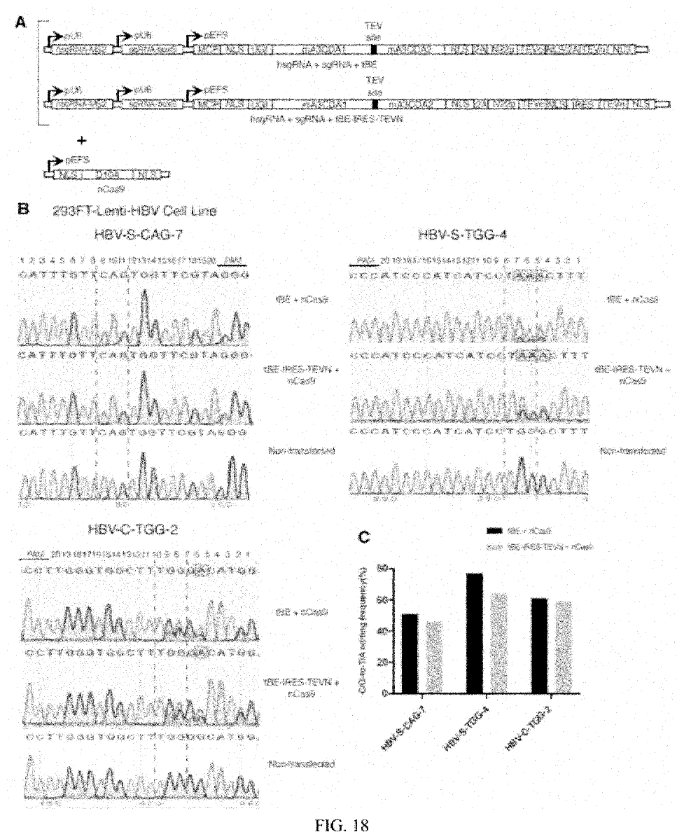

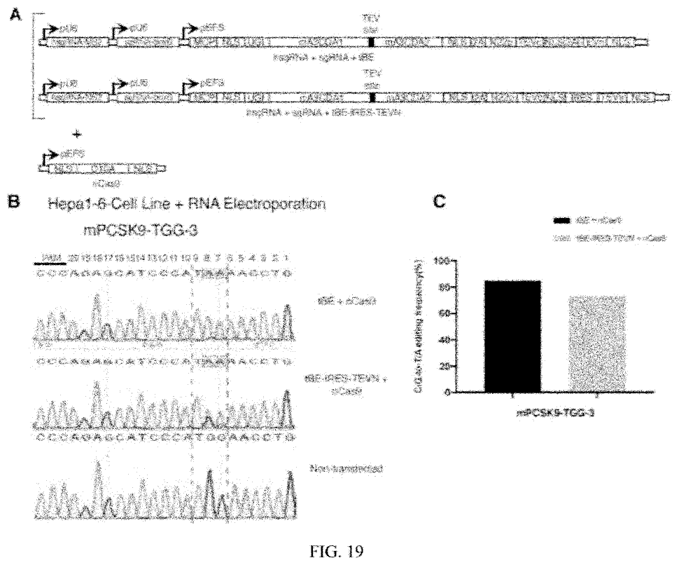

It has been found that either of these two 2A peptides can be replaced by the internal ribosome entry site (IRES). Both tBE-IRES-TEVC and tBE-IRES-TEVN induced effective base editing at human genomic sites (FIGS. 14-16). In addition, the tBE-IRES-TEVN also induced precise gene editing at HBV virus genomic sites (FIGS. 17 and 18) and mouse genomic sites (FIG. 19).

The present disclosure is not to be limited in scope by the specific embodiments described which are intended as single illustrations of individual aspects of the disclosure, and any compositions or methods which are functionally equivalent are within the scope of this disclosure. It will be apparent to those skilled in the art that various modifications and variations can be made in the methods and compositions of the present disclosure without departing from the spirit or scope of the disclosure. Thus, it is intended that the present disclosure cover the modifications and variations of this disclosure provided they come within the scope of the appended claims and their equivalents.

All publications and patent applications mentioned in this specification are herein incorporated by reference to the same extent as if each individual publication or patent application was specifically and individually indicated to be incorporated by reference.

1. A protein, comprising a catalytic domain of a mutant mouse APOBEC3 protein, wherein the catalytic domain has at least 85% sequence identity to amino acid residues 35-141 of SEQ ID NO: 1 and comprises a substitution, relative to SEQ ID NO: 1, at a residue selected from the group consisting of Y35, K37, R39, K40, N66, W102, Y132, and combinations thereof.

2. The protein of claim 1, wherein the substitution is selected from the group consisting of:

| Residue | Substitution |
|---|---|
| Y35 | D or E |
| K37 | D or E |
| R39 | A, G, I, L or V |
| K40 | A, G, I, L, V or H |
| N66 | G, A, I, L, V or Q |
| W102 | Y or F, and |
| Y132 | F or W. |

3. The protein of claim 1, wherein the catalytic domain retains the amino acids of SEQ ID NO: 1 at residues H71 and E73.

4. The protein of claim 1, wherein the catalytic domain retains the amino acids of SEQ ID NO: 1 at residues D41, F43, F64, A72, P104, C105 and C108.

5. The protein of claim 1, wherein the substitution is selected from the group consisting of Y35D, Y35E, K37D, R39A, K40A, K40H, N66A, N66G, N66Q, W102Y, W102F, Y132F, and combinations thereof.

6. The protein of claim 5, wherein the substitution is Y35D or Y35E.

7. The protein of claim 1, wherein the catalytic domain comprises the amino acid sequence of SEQ ID NO: 3.

8. The protein of claim 5, wherein the substitution is K40H and W102Y.

9. The protein of claim 1, wherein the catalytic domain comprises the amino acid sequence of SEQ ID NO: 5.

10. A fusion protein comprising:
a first fragment comprising the protein of claim 1, and
a second fragment comprising a nucleobase deaminase inhibitor.

11. The fusion protein of claim 10, further comprising a protease cleavage site between the first fragment and the second fragment.

12. The fusion protein of claim 10, wherein the nucleobase deaminase inhibitor is an inhibitory domain of a nucleobase deaminase.

13. The fusion protein of claim 10, wherein the nucleobase deaminase inhibitor comprises the amino acid sequence of SEQ ID NO: 7, 8 or 9, or amino acids residues 128-223 of SEQ ID NO: 7.

14. A dual guide RNA system, comprising:
a target single guide RNA comprising a first spacer having sequence complementarity to a target nucleic acid sequence proximate to a first PAM site,
a helper single guide RNA comprising a second spacer having sequence complementarity to a second nucleic acid sequence proximate to a second PAM site,
a clustered regularly interspaced short palindromic repeats (CRISPR)-associated (Cas) protein, and
a protein of claim 1.

15. The dual guide RNA system of claim 14, wherein the second PAM site is from 34 to 91 bases from the first PAM site.

16. A method for introducing a C-to-T substitution at a cytosine in a target nucleic acid, comprising contacting the target nucleic acid with a CRISPR-associated (Cas) protein, a protein of claim 1, a single-guide RNA (sgRNA), and a helper single-guide RNA (hsgRNA), wherein the sgRNA and the hsgRNA can hybridize to the target nucleic acid.

17. (canceled)

18. (canceled)

19. A method for introducing a C-to-T substitution at a cytosine in a target nucleic acid, comprising contacting the target nucleic acid with a CRISPR-associated (Cas) protein, a fusion protein of claim 10, a single-guide RNA (sgRNA), and a helper single-guide RNA (hsgRNA), wherein the sgRNA and the hsgRNA can hybridize to the target nucleic acid, wherein cytosine is between nucleotide positions 6 and 8 3′ to a protospacer adjacent motif (PAM) sequence on the target nucleic acid sequence, and wherein the catalytic domain comprises the amino acid sequence of SEQ ID NO: 3.

20. A method for introducing a C-to-T substitution at a cytosine in a target nucleic acid, comprising contacting the target nucleic acid with a CRISPR-associated (Cas) protein, a fusion protein of claim 10, a single-guide RNA (sgRNA), and a helper single-guide RNA (hsgRNA), wherein the sgRNA and the hsgRNA can hybridize to the target nucleic acid, wherein cytosine is between nucleotide positions 4 and 8 3′ to a protospacer adjacent motif (PAM) sequence on the target nucleic acid sequence, and wherein the catalytic domain comprises the amino acid sequence of SEQ ID NO: 5.

21. A base editing system, comprising:
(a) a first fusion protein comprising a nucleobase deaminase, a nucleobase deaminase inhibitor, and a first RNA recognition peptide, wherein the nucleobase deaminase and the nucleobase deaminase inhibitor is separated by a protease cleavage site that can be cleaved by a protease;
(b) a second fusion protein comprising an inactive portion of the protease fused to a second RNA recognition peptide that is different from the first RNA recognition peptide;
(c) a second portion of the protease which, in combination with the first portion, can carry out the protease activity to cleave the protease cleavage site;
(d) a helper sgRNA further comprising a first RNA recognition site recognizable by first RNA recognition peptide;
(e) a sgRNA further comprising a second RNA recognition site recognizable by the second RNA recognition peptide; and
(f) a Cas protein,
wherein the nucleobase deaminase is a protein of claim 1.

22. (canceled)

23. (canceled)

24. A polynucleotide, comprising
a first fragment encoding (a) a first fusion protein comprising a nucleobase deaminase, a nucleobase deaminase inhibitor, and a first RNA recognition peptide, wherein the nucleobase deaminase and the nucleobase deaminase inhibitor is separated by a protease cleavage site that can be cleaved by a protease;
a second fragment encoding (b) a second fusion protein comprising an inactive portion of the protease fused to a second RNA recognition peptide that is different from the first RNA recognition peptide;
a third fragment encoding (c) a second portion of the protease which, in combination with the first portion, can carry out the protease activity to cleave the protease cleavage site;
a fourth fragment encoding (d) a helper sgRNA further comprising a first RNA recognition site recognizable by first RNA recognition peptide; and
a fifth fragment encoding (e) a sgRNA further comprising a second RNA recognition site recognizable by the second RNA recognition peptide,
wherein the first, second, and third fragments are configured to be transcribed into a single mRNA molecule, wherein
(1) the first and second fragments are separated by a first separating sequence encoding a first internal ribosome entry site (IRES), and the second and third fragments are separated by a second separating sequence encoding a first self-cleavage peptide; or
(2) the first and second fragments are separated by a first separating sequence encoding a second self-cleavage peptide, and the second and third fragments are separated by a second separating sequence encoding a second internal ribosome entry site (IRES).

25. (canceled)
